(19) United States
(12) Patent Application Publication
(10) Pub. No.: US 2024/0285419 A1
(43) Pub. Date: Aug. 29, 2024

(54) JOINT DEVICE

(71) Applicant: HONDA MOTOR CO., LTD., Tokyo (JP)

(72) Inventor: Kei SHIMADA, Saitama (JP)

(73) Assignee: HONDA MOTOR CO., LTD., Tokyo (JP)

(21) Appl. No.: 18/568,687

(22) PCT Filed: Jun. 8, 2022
(86) PCT No.: PCT/JP2022/023174

§ 371 Date: Dec. 8, 2023

(30) Foreign Application Priority Data
Jun. 11, 2021 (JP) 2021-098286

(51) Int. Cl. A61F 2/64; A61F 2/68; A61F 2/70

(52) U.S. Cl. CPC A61F 2/64; A61F 2/70; A61F 2002/6836

(57) ABSTRACT

An electric prosthetic leg includes a below-knee member, an above-knee member, a knee joint mechanism coupling the below-knee member and the above-knee member such that angles formed between the below-knee member and the above-knee member are variable, and an expansion-contraction device capable of varying the angles formed between the below-knee member and the above-knee member by expanding and contracting, in which one side of the expansion-contraction device in an extending direction is mechanically connected to the below-knee member, and the other side of the expansion-contraction device is mechanically connected to the above-knee member. Assuming that smaller one of minimum formed angles, of the angles formed between the below-knee member and the above-knee member centering on a pivoting portion of the knee joint mechanism, is defined as a second formed angle, the expansion-contraction device is partially disposed on a side of a first formed angle.

### TECHNICAL FIELD

The present invention relates to a joint device.

### BACKGROUND ART

In the related art, as a joint device used for a coupling portion coupling two members, there is a joint device including an expansion-contraction device capable of changing an angle formed by the two members. An example of such a joint device is a prosthetic leg used for a knee joint. Patent Literature 1 discloses that a sensor for detecting contraction motion of a muscle at a cut-off end portion of an amputated leg is provided in a thigh socket of a prosthetic leg attached to the cut-off end portion of the amputated leg, and a throttling condition of a variable valve of a hydraulic cylinder for adjusting resistance of bending and stretching of a knee joint portion is controlled based on detection information from the sensor.

### CITATION LIST

### Patent Literature

Patent Literature 1: JPH11-019105A

### SUMMARY OF INVENTION

### Technical Problem

However, in the prosthetic leg described in Patent Literature 1, although resistance of bending and stretching can be generated, power of bending and stretching cannot be generated. Particularly, in order to smoothly ascend steps, it is necessary to stretch the knee joint in a state where a load is applied.

The present invention provides a joint device capable of stretching and bending a coupling portion by an expansion-contraction device.

### Solution to Problem

The present invention is a joint device, including:
a first member;
a second member;
a coupling portion configured to couple the first member and the second member such that angles formed between the first member and the second member are variable; and
an expansion-contraction device capable of varying the angles formed between the first member and the second member by expanding and contracting, in which one side of the expansion-contraction device in an extending direction is mechanically connected to the first member, and an other side of the expansion-contraction device is mechanically connected to the second member,
in which assuming that one side of one circumference centering on a coupling axis of the coupling portion and having angles formed between the first member and the second member is defined as a first formed angle, and an other side of the one circumference is defined as a second formed angle, and
smaller one of minimum formed angles, of the first formed angle and the second formed angle, formed in a range of relative movement between the first member and the second member is defined as the second formed angle,
at least a part of the expansion-contraction device is disposed on a side of the first formed angle.

The present invention is a joint device, including:
a first member;
a second member;
a coupling portion configured to couple the first member and the second member such that angles formed between the first member and the second member are variable; and
an expansion-contraction device capable of varying the angles formed between the first member and the second member by expanding and contracting, in which one side of the expansion-contraction device in an extending direction is mechanically connected to the first member, and an other side of the expansion-contraction device is mechanically connected to the second member,
in which the expansion-contraction device has a motion conversion mechanism including a shaft member and a cylindrical member that performs translational motion along an axis of the shaft member due to a rotation of the shaft member, and
in which assuming that one side of one circumference centering on a coupling axis of the coupling portion and having angles formed between the first member and the second member is defined as a first formed angle, and an other side of the one circumference is defined as a second formed angle, and
smaller one of minimum formed angles, of the first formed angle and the second formed angle, formed in a range of relative movement between the first member and the second member is defined as the second formed angle.
the expansion-contraction device is provided such that a tensile force acts on the shaft member as the second formed angle increases.

### Advantageous Effects of Invention

According to the present invention, the coupling portion can be stretched and bent by the expansion-contraction device.

### BRIEF DESCRIPTION OF DRAWINGS

FIG. 1 is a perspective view of an electric prosthetic leg according to a first embodiment of the present invention as viewed obliquely from a front side.

FIG. 2 is a diagram showing a power transmission unit of the electric prosthetic leg shown in FIG. 1.

FIG. 3 is a diagram showing a first transmission state in which in the power transmission unit shown in FIG. 2, a first intermittence unit of a first intermittence mechanism is in a forced free state and a third intermittence unit of a second intermittence mechanism is in a power transmittable state.

FIG. 4 is a diagram showing a second transmission state in which in the power transmission unit shown in FIG. 2, the first intermittence unit of the first intermittence mechanism is in a power transmittable state and the third intermittence unit of the second intermittence mechanism is in a forced free state.

FIG. 5 is a cross-sectional view taken along a line A-A in FIG. 3.

FIG. 6 is a cross-sectional view taken along a line B-B in FIG. 3.

FIG. 7 is a perspective view of a cross section taken along a line C-C in FIG. 3.

FIG. 8 is a perspective view of a cross section taken along a line D-D in FIG. 3.

FIG. 9 is a diagram showing movements (A) to (F) of a human and an electric prosthetic leg during step ascending.

FIG. 10 is a diagram illustrating power for stretching a knee joint mechanism from a bent state (from (A) to (B) in FIG. 9) during step ascending.

FIG. 11 is a diagram illustrating power for bending the knee joint mechanism from a stretched state (from (D) to (E) in FIG. 9) during step ascending.

FIG. 12 is a diagram showing movements of a human and an electric prosthetic leg during step ascending, walking on level ground, and step descending.

FIG. 13 is a diagram illustrating power for bending the knee joint mechanism from a stretched state while attenuating an external force during step descending or walking on level ground.

FIG. 14 is a diagram showing an electric prosthetic leg according to a modification, and illustrating power for stretching a knee joint mechanism from a bent state while attenuating an external force during walking on level ground.

FIG. 15 is a diagram showing a power transmission unit of an electric prosthetic leg according to a second embodiment of the present invention.

FIG. 16 is a diagram showing a power transmission unit of an electric prosthetic leg according to a third embodiment of the present invention.

FIG. 17 is a cross-sectional view of a two-way clutch.

FIG. 18 is a perspective view showing an example of a retainer shown in FIG. 17 (including a roller, guides, and rubber balls),

FIG. 19 is a perspective view showing another example of the retainer shown in FIG. 17 (including a roller, guides, and O-rings).

FIG. 20 is a diagram showing an operation of a second operating mechanism 240 in a second intermittence unit and a fourth intermittence unit shown in FIG. 16, in which (A) shows a state where the second intermittence unit and the fourth intermittence unit are in an off state, and (B) shows a state where the second intermittence unit is in an on state and the fourth intermittence unit is in an off state, and (C) shows a state where the second intermittence unit is in an off state and the fourth intermittence unit is in an on state.

FIG. 21 shows (A) a cross-sectional view taken along a line A-A in FIG. 16 showing that the second intermittence unit is in the off state, and (B) a position of a second operating rod 241 in this case.

FIG. 22 shows (A) a cross-sectional view taken along the line A-A in FIG. 16 showing that the second intermittence unit is operated from the off state to the on state, and (B) a position of the second operating rod 241 in this case.

FIG. 23 shows (A) a cross-sectional view taken along the line A-A in FIG. 16 showing a forward rotation on state of the second intermittence unit shown in FIG. 16, and (B) a position of the second operating rod 241 in this case.

FIG. 24 shows (A) a cross-sectional view taken along the line A-A in FIG. 16 showing a rearward rotation on state of the second intermittence unit shown in FIG. 16, and (B) a position of the second operating rod 241 in this case.

FIG. 25 shows (A) a cross-sectional view taken along the line A-A in FIG. 16 showing the forward rotation on state of the second intermittence unit shown in FIG. 16, and (B) a position of the second operating rod 241 in this case.

FIG. 26 shows (A) a cross-sectional view taken along the line A-A in FIG. 16 showing that the second intermittence unit shown in FIG. 16 is operated from the on state to the off state, and (B) a position of the second operating rod 241 in this case.

FIG. 27 is a perspective view of an electric prosthetic leg according to a fourth embodiment of the present invention as viewed obliquely from a front side.

FIG. 28 is an exploded perspective view of the electric prosthetic leg shown in FIG. 27.

FIG. 29 is cross-sectional view of the electric prosthetic leg shown in FIG. 27.

FIG. 30 is a cross-sectional view showing a main part of the electric prosthetic leg shown in FIG. 27 in a stretching state.

FIG. 31 is a cross-sectional view showing a main part of the electric prosthetic leg shown in FIG. 27 in a bending state.

FIG. 32 is a cross-sectional view showing a main part of the electric prosthetic leg shown in FIG. 27 in a maximum bent state.

FIG. 33 is a diagram showing the maximum bent state of the electric prosthetic leg shown in FIG. 27, and illustrating angles formed between an above-knee member 120 and a below-knee member 110 and a load acting on a spindle unit SP.

### DESCRIPTION OF EMBODIMENTS

Hereinafter, an electric prosthetic leg as an embodiment of a joint device of the present invention will be described below with reference to the drawings. Note that in the following description, a front-rear direction, a left-right direction, and an upper-lower direction are defined with reference to a user of the electric prosthetic leg. In the drawings, a front side of the electric prosthetic leg is denoted by Fr, a rear side is denoted by Rr, a left side is denoted by L, a right side is denoted by R, an upper side is denoted by U, and a lower side is denoted by D.

### [Electric Prosthetic Leg]

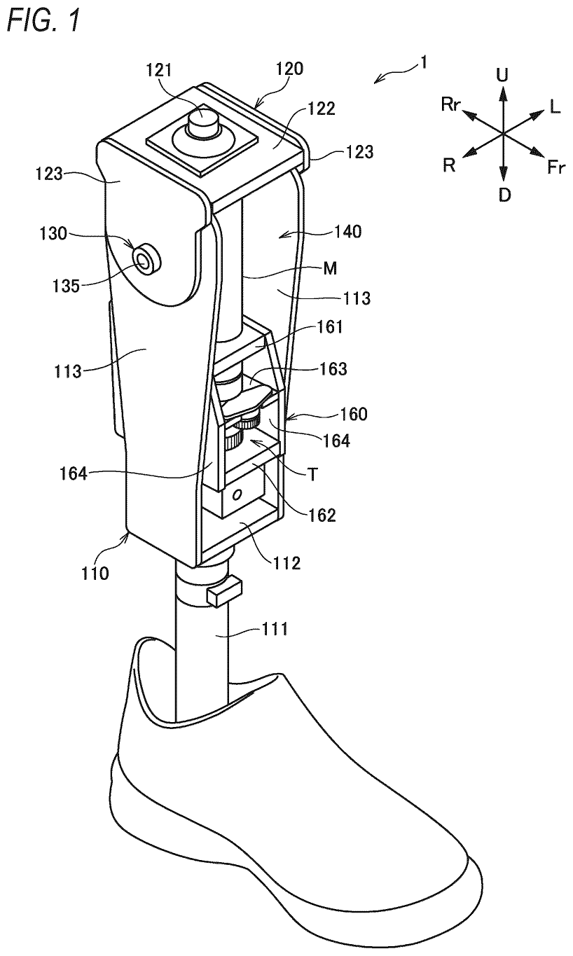

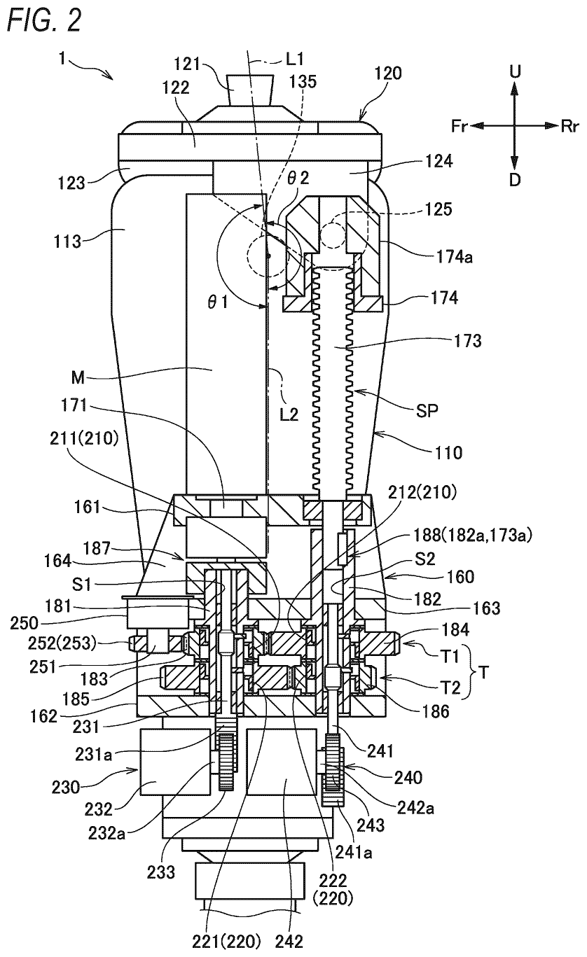

As shown in FIGS. 1 and 2, an electric prosthetic leg 1 according to a first embodiment is a prosthetic leg that is attached to a leg portion of a person who does not have a knee. The electric prosthetic leg 1 includes: a below-knee member 110 positioned on a lower side of the knee, an above-knee member 120 positioned on an upper side of the knee and attached to a thigh portion, a knee joint mechanism 130 that couples the below-knee member 110 and the above-knee member 120 such that angles formed between the below-knee member 110 and the above-knee member 120 are variable, an expansion-contraction device 140 capable of varying the angles formed between the below-knee member 110 and the above-knee member 120 by expanding and contracting, and a battery (not shown).

The above-knee member 120 includes an upper wall portion 122 provided with an adapter 121 connected to a socket (not shown), and a pair of upper side wall portions 123 extending downward from left and right ends of the upper wall portion 122, respectively, and the above-knee member 120 has a substantially U-shape with a lower opening when viewed from the front-rear direction.

The below-knee member 110 includes a lower wall portion 112 provided with a leg portion 111, and a pair of lower side wall portions 113 extending upward from left and right ends of the lower wall portion 112, respectively, and the below-knee member 110 has a substantially U-shape with an upper opening when viewed in the front-rear direction.

The pair of lower side wall portions 113 of the below-knee member 110 are coupled between the pair of upper side wall portions 123 of the above-knee member 120 in a manner of being rotatable around a pivoting portion 135. With this mechanism, the knee joint mechanism 130 is configured to couple the below-knee member 110 and the above-knee member 120 such that the angles formed between the below-knee member 110 and the above-knee member 120 are variable.

The expansion-contraction device 140 capable of changing the angles formed between the above-knee member 110 and the below-knee member 120 is provided in a space formed between the below-knee member 120 and the above-knee member 110. The expansion-contraction device 140 extends in the upper-lower direction, which will be described later in detail, and is mechanically connected to the above-knee member 120 on one side in the extending direction and mechanically connected to the below-knee member 110 on the other side in the extending direction. Note that the term “mechanically connected” is a concept that includes a configuration of direct connection and a configuration of connection via another member.

The expansion-contraction device 140 includes: a motor M that outputs rotational power, a transmission T that transmits the power of the motor M, a spindle unit SP that is connected to the transmission T in a manner of being capable of transmitting power and converts the rotational power output from the transmission T into translational motion, a first intermittence mechanism 210 and a second intermittence mechanism 220 provided in the transmission T, a first operating mechanism 230 and a second operating mechanism 240 for switching between the first intermittence mechanism 210 and the second intermittence mechanism 220, and a rotary damper 250 that attenuates an external force input from the spindle unit SP.

The transmission T includes a top plate portion 161, a bottom plate portion 162, a middle plate portion 163 displaced in parallel with and between the top plate portion 161 and the bottom plate portion 162, and a pair of side plate portions 164 coupling left and right ends of the top plate portion 161, the bottom plate portion 162 and the middle plate portion 163, and the transmission T is provided with a transmission case 160 having a rectangular shape when viewed from the front-rear direction. The transmission case 160 is swingably and immovably supported by the below-knee member 110 via a lower swinging portion (not shown). That is, in the expansion-contraction device 140, the transmission case 160 is mechanically connected to the below-knee member 110 at the lower swinging portion (not shown).

The motor M is disposed forward and upward of the top plate portion 161 of the transmission case 160 such that an output shaft 171 protrudes into the transmission case 160 through the top plate portion 161. The spindle unit SP is disposed on an opposite side of the motor M in the front-rear direction. The spindle unit SP includes a male-threaded spindle 173 and a female-threaded sleeve 174, and rotation of the spindle 173 causes the sleeve 174 to perform translational motion along an axis of the spindle 173 (a line that passes through the axis and extends in an extending direction thereof).

In the present embodiment, the spindle 173 performs rotational motion by receiving the rotational power of the motor M transmitted by the transmission T. On the other hand, in the sleeve 174, a base portion 174a of the sleeve 174 is attached to a pair of inner side wall portions 124 extending downward from the upper wall portion 122 of the above-knee member 120 in a manner of being swingable and immovable centering on an upper swing portion 125. That is, in the expansion-contraction device 140, the base portion 174a of the sleeve 174 is mechanically connected to the above-knee member 120 at the upper swing portion 125. Therefore, when the spindle 173 rotates to one side by receiving the rotational power of the motor M transmitted by the transmission T, the sleeve 174 is translated away from the transmission T, and when the spindle 173 rotates to the other side, the sleeve 174 is translated in a direction of approaching the transmission T. Note that the translational movement of the sleeve 174 away from the transmission T may be referred to as an expanding operation of the spindle unit SP, and conversely, the translational movement of the sleeve 174 approaching the transmission T may be referred to as a contracting operation of the spindle unit SP.

That is, a distance between the sleeve 174 and the transmission T increases or decreases depending on a rotation direction of the spindle 173. Since the sleeve 174 is immovably attached to the above-knee member 120 as described above, the distance between the sleeve 174 and the transmission T increases or decreases depending on the rotation direction of the spindle 173, and therefore, the below-knee member 110 to which the transmission T is attached and the above-knee member 120 to which the sleeve 174 is attached rotate around the pivoting portion 135. Accordingly, the angles formed between the above-knee member 120 and the below-knee member 110 vary.

Here, the angles formed between the above-knee member 120 and the below-knee member 110 are angles defined by a first virtual line L1 connecting a center of the pivoting portion 135 of the knee joint mechanism 130 and the adapter 121 of the above-knee member 120 and a second virtual line L2 that extends downward in a vertical direction through the center of the pivoting portion 135 of the knee joint mechanism 130 and the below-knee member 110. One side of one circumference centering on the pivoting portion 135 of the knee joint mechanism 130 and having angles formed between the below-knee member 110 and the above-knee member 120 is defined as a first formed angle θ1, and the other side of the one circumference is defined as a second formed angle θ2. When a smaller one of minimum formed angles, of the first formed angle θ1 and the second formed angle θ2, formed in a range of relative movement between the below-knee member 110 and the above-knee member 120 is defined as the second formed angle θ2, the angle formed on a back side of a knee of a user of the electric prosthetic leg 1 is defined as the second formed angle θ2. The first formed angle θ1 takes a value of approximately 170° to 310°, and the second formed angle θ2 takes a value of approximately 50° to 190°.

FIG. 2 shows a stretched state of the knee joint mechanism 130, in which the first formed angle θ1 is approximately 170°, and the second formed angle θ2 is approximately 190°. In the electric prosthetic leg 1 of the present embodiment, at least a part of the expansion-contraction device 140 is provided on the first formed angle θ1 side (shin side) with respect to the second virtual line L2. More specifically, in the present embodiment, the motor M of the expansion-contraction device 140 is provided on the first formed angle θ1 side (shin side) with respect to the second virtual line L2. On the other hand, the spindle unit SP of the expansion-contraction device 140 is provided on the second formed angle θ2 side (calf side) with respect to the second virtual line L2. Therefore, when the second formed angle θ2 becomes smaller and the first formed angle θ1 becomes larger, a length of the spindle unit SP is reduced and the knee joint mechanism 130 is bent. On the other hand, when the second formed angle θ2 becomes larger and the first formed angle θ1 becomes smaller, the length of the spindle unit SP increases and the knee joint mechanism 130 is stretched. As a result, a setting range of the minimum formed angle of the second formed angle θ2 can be made smaller than when both the motor M and the spindle unit SP are disposed on the second formed angle θ2 side. Therefore, the user of the electric prosthetic leg 1 can bend the knee more deeply.

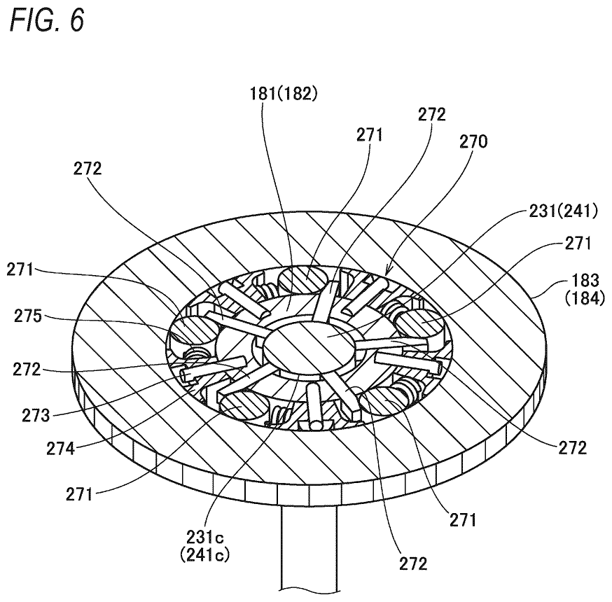

As shown in FIGS. 2 to 6, the transmission T includes a first transmission mechanism T1 that transmits the power of the motor M to the spindle unit SP at a first transmission ratio, and a second transmission mechanism T2 that transmits the power of the motor M to the spindle unit SP at a second transmission ratio, which is different from the first transmission ratio. The first transmission mechanism T1 is switched between a power cutoff state and a power connection state by the first intermittence mechanism 210, and the second transmission mechanism T2 is switched between a power cutoff state and a power connection state by the second intermittence mechanism 220.

According to such a transmission T, by providing two power transmission paths with different transmission ratios, it is possible to switch an operating speed and generated power for stretching and bending in the knee joint mechanism 130. One of the first transmission mechanism T1 and the second transmission mechanism T2 may be a speed reduction mechanism and the other may be a speed increasing mechanism, or one may be a constant speed mechanism and the other may be a speed reduction mechanism or a speed increasing mechanism, or both may be speed reduction mechanisms, or both may be speed increasing mechanisms, as long as the first transmission ratio is different from the second transmission ratio.

The first transmission ratio is a post-transmission rotation speed, which is a rotation speed on a side opposite to the motor M (spindle unit SP side) in the first transmission mechanism T1, with respect to a pre-transmission rotation speed, which is a rotation speed on the motor M side in the first transmission mechanism T1. The second transmission ratio is a post-transmission rotation speed, which is a rotation speed on the side opposite to the motor M (spindle unit SP side) in the second transmission mechanism T2, with respect to a pre-transmission rotation speed, which is a rotation speed on the motor M side in the second transmission mechanism T2.

For example, when the first transmission ratio of the first transmission mechanism T1 is smaller than 1, the rotation speed on the side opposite to the motor M (spindle unit SP side) becomes lower than the rotation speed on the motor M side, and a torque increases. When the second transmission ratio of the second transmission mechanism T2 is greater than 1, the rotation speed on the side opposite to the motor M (spindle unit SP side) becomes higher than the rotation speed on the motor M side, and a torque decreases. In the present embodiment, the first transmission ratio is set to be smaller than 1, and the second transmission ratio is set to be greater than 1, and a diameter of a first drive gear 183 is smaller than that of a second drive gear 185. Note that in the present embodiment, the first transmission mechanism T1 is disposed above the second transmission mechanism T2.

The first transmission mechanism T1 and the second transmission mechanism T2 include a first shaft 181 rotatably disposed on a downward extension line of the output shaft 171 of the motor M, and a second shaft 182 rotatably disposed on a downward extension line of the spindle 173 of the spindle unit SP. The first shaft 181 is integrally rotatably coupled to the output shaft 171 of the motor M via a coupling 187 that allows an axial center error, and the second shaft 182 is integrally rotatably connected to the spindle 173 of the spindle unit SP via a key 188 and key grooves 182a and 173a. Note that the output shaft 171 of the motor M and the first shaft 181 may be coupled by key fitting or spline fitting without using the coupling 187. The spindle 173 of the spindle unit SP and the second shaft 182 may be coupled by spline fitting or a coupling instead of key fitting.

The first transmission mechanism T1 includes the first drive gear 183 and a first driven gear 184 that mesh with each other. The first drive gear 183 is relatively rotatably supported by the first shaft 181, and the first driven gear 184 is relatively rotatably supported by the second shaft 182. The first transmission mechanism T1 of the present embodiment is a deceleration transmission mechanism in which the first drive gear 183 has a diameter smaller than that of the first driven gear 184, and can make the spindle unit SP expand and contract at low speed and high torque.

The second transmission mechanism T2 includes the second drive gear 185 and a second driven gear 186 that mesh with each other. The second drive gear 185 is relatively rotatably supported by the first shaft 181, and the second driven gear 186 is relatively rotatably supported by the second shaft 182. The second transmission mechanism T2 of the present embodiment is an acceleration transmission mechanism in which the second drive gear 185 has a diameter greater than that of the second driven gear 186, and can make the spindle unit SP expand and contract at high speed and low torque.

The first intermittence mechanism 210 includes a first intermittence unit 211 provided between the first drive gear 183 and the first shaft 181 and a second intermittence unit 212 provided between the first driven gear 184 and the second shaft 182.

The second intermittence mechanism 220 includes a third intermittence unit 221 provided between the second drive gear 185 and the first shaft 181 and a fourth intermittence unit 222 provided between the second driven gear 186 and the second shaft 182.

These intermittence units 211, 212, 221, and 222 have a common configuration, and are configured to be switchable between a cutoff state in which power transmission is cut off, and a power transmittable state in which rotational power in both one direction and the other direction can be transmitted.

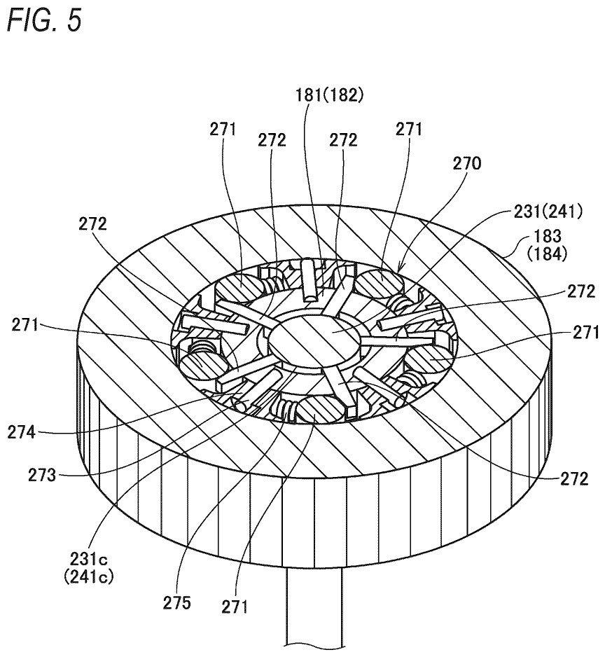

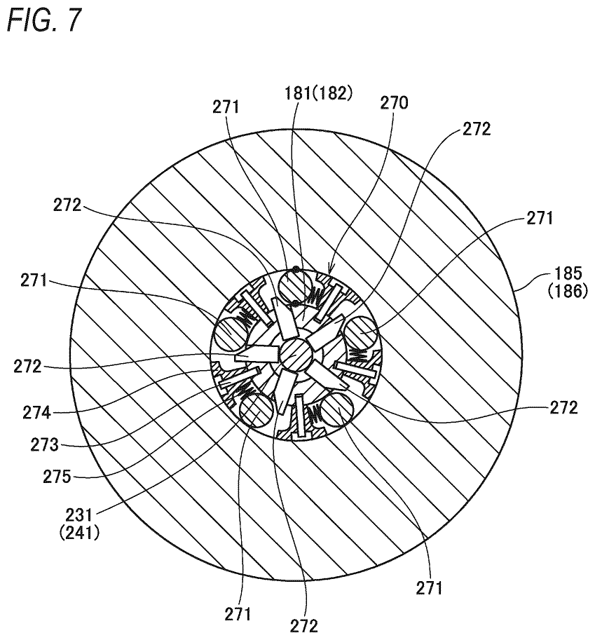

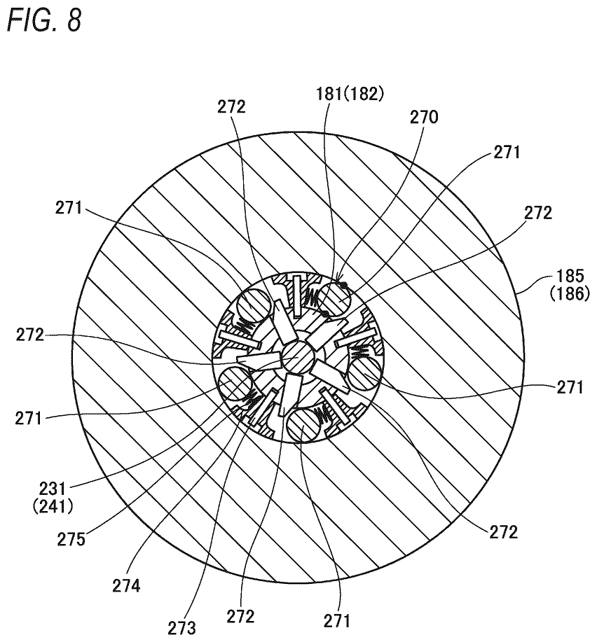

Each of the intermittence units 211, 212, 221, and 222 of the present embodiment is configured by combining two one-way clutches 270 with a forced free function, as shown in FIGS. 5 to 8. Each one-way clutch 270 is disposed between outer peripheral surface portions of the shafts 181 and 182 and inner peripheral surface portions of the gears 183 to 186, and includes a plurality of rollers 271 that are in an engaged state when rotational power in one direction is input from the shaft side or gear side so as to transmit the rotational power, and in a disengaged state when rotational power in the other direction is input from the shaft side or gear side so as to cut off the rotational power; a retainer 274 that holds the plurality of rollers 271 at predetermined intervals; a plurality of pins 272 that forcibly hold the plurality of rollers 271 in a disengaged position to cut off the rotational power in one direction and the other direction. Note that in the drawings, the reference numeral 273 denotes a fixing pin that fixes the retainer 274 to the shafts 181 and 182, and the reference numeral 275 denotes a spring that biases the roller 271 from the retainer 274 side toward the pin 272 side. Each of the intermittence units 211, 212, 221, and 222 is configured by overlapping two one-way clutches 270 such that the rotation directions of transmission are reversed. According to these intermittence units 211, 212, 221, and 222, it is possible to switch between a cutoff state in which the two one-way clutches 270 are forcibly freed to cut off the power transmission, and a power transmittable state in which either one of the two one-way clutches 270 is in an engaged state and can transmit rotational power in both one direction and the other direction.

The first operating mechanism 230 includes a first operating rod 231 that is provided to operate the pin 272 of the first intermittence unit 211 of the first intermittence mechanism 210 and the pin 272 of the third intermittence unit 221 of the second intermittence mechanism 220, and a first servomotor 232 that linearly moves the first operating rod 231. Note that the operating rod provided to operate the pin 272 of the first intermittence unit 211 and the operating rod provided to operate the pin 272 of the third intermittence unit 221 may be different, and a servomotor may be provided to linearly move each operating rod.

The second operating mechanism 240 includes a second operating rod 241 that is provided to operate the pin 272 of the second intermittence unit 212 of the first intermittence mechanism 210 and the pin 272 of the fourth intermittence unit 222 of the second intermittence mechanism 220, and a second servomotor 242 that linearly moves the second operating rod 241. Note that the operating rod provided to operate the pin 272 of the second intermittence unit 212 and the operating rod provided to operate the pin 272 of the fourth intermittence unit 222 may be different, and a servomotor ma be provided to linearly move each operating rod.

The first shaft 181 is a hollow shaft having a first internal space S1 and extending in a direction of a rotational axis, and the second shaft 182 is a hollow shaft having a second internal space S2 and extending in a direction of a rotational axis. The first operating rod 231 is disposed in a manner of being movable up and down in the first internal space S1, and the second operating rod 241 is disposed in a manner of being movable up and down in the second internal space S2. The first shaft 181 and the second shaft 182 are disposed in a manner of extending in the vertical direction when the user of the electric prosthetic leg 1 stands upright.

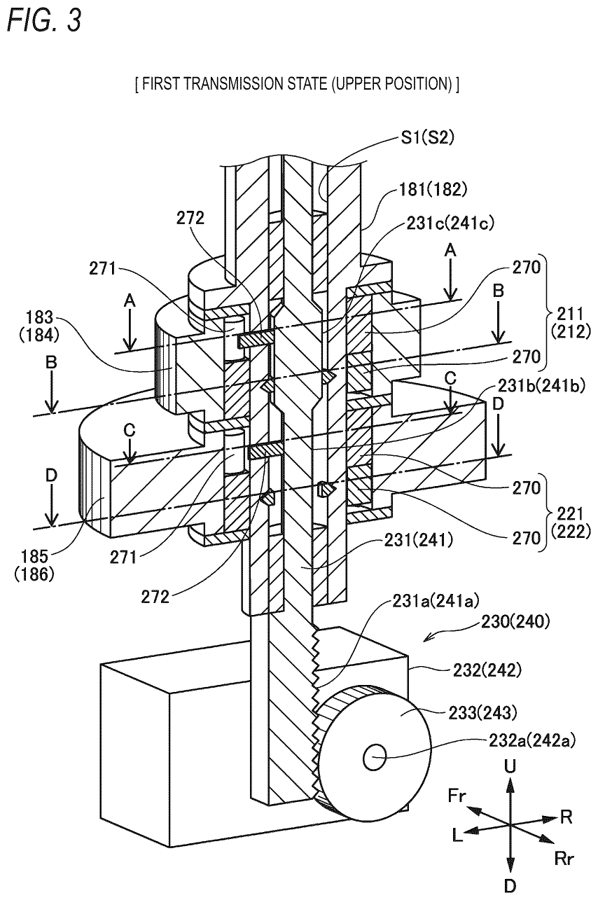

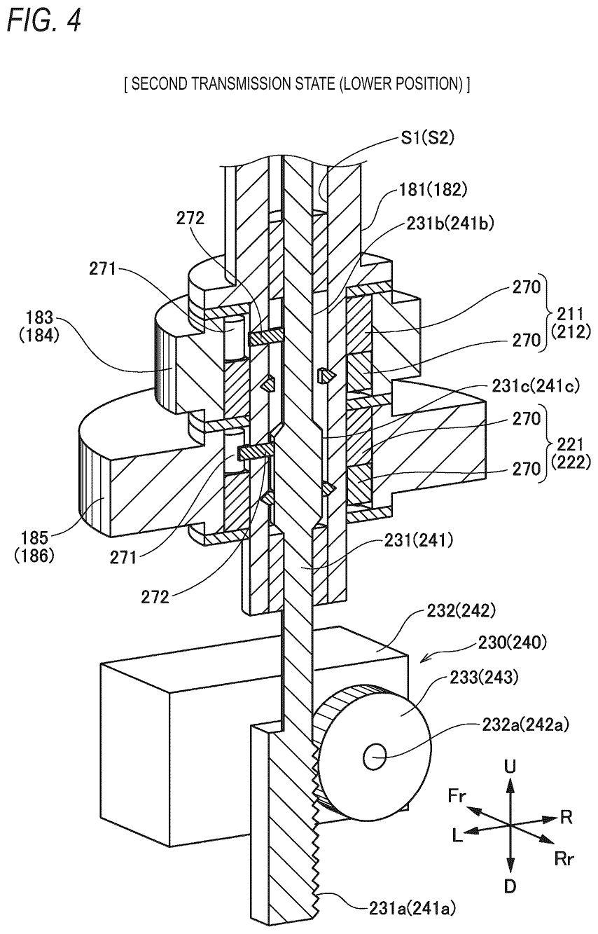

The first operating rod 231 includes a rack 231a on a lower end side. A pinion 233 provided on an output shaft 232a of the first servomotor 232 meshes with the rack 231a, and a position of the first operating rod 231 is switched between an upper position shown in FIG. 3 and a lower position shown in FIG. 4 according to drive of the first servomotor 232. Note that although FIGS. 3 to 6 show the first operating mechanism 230, the second operating mechanism 240 also has a similar configuration. Reference numerals in parentheses in FIGS. 3 to 8 indicate each component of the second operating mechanism 240 that corresponds to each component of the first operating mechanism 230.

The second operating rod 241 includes a rack 241a on a lower end side. A pinion 243 provided on an output shaft 242a of the second servomotor 242 meshes with the rack 241a, and a position of the second operating rod 241 is switched between an upper position and a lower position according to drive of the second servomotor 242.

The pins 272 of each intermittence unit 211, 212, 221, 222 are provided in a manner of being movable in a radial direction with respect to the rotational axes of the first shaft 181 and the second shaft 182, and the first operating rod 231 and the second operating rod 241 are provided such that outer peripheral portions thereof abut against inner end portions of the pins 272. The outer peripheral portions of the first operating rod 231 and the second operating rod 241 includes small diameter portions 231b and 241b that position the pins 272 in a forced free release position, and large diameter portions 231c and 241 c that push the pins 272 outward to a forced free position. Note that between the small diameter portions 231b, 241b and the large diameter portions 231c, 241c, sloped portions are provided to connect the small diameter portions 231b, 241b and the large diameter portions 231c, 241c without steps.

The present embodiment has a first transmission state in which the first operating rod 231 and the second operating rod 241 are in the upper position, and a second transmission state in which the first operating rod 231 and the second operating rod 241 are in the lower position. In the first transmission state, as shown in FIGS. 3 and 5 to 8, the large diameter portions 231c and 241c of the first operating rod 231 and the second operating rod 241 forcefully free the first intermittence unit 211 and the second intermittence unit 212 of the first intermittence mechanism 210, so that the motor M and the spindle unit SP are in the power transmission state via the second transmission mechanism T2. In the second transmission state, as shown in FIG. 4, the large diameter portions 231c and 241c of the first operating rod 231 and the second operating rod 241 forcefully free the third intermittence unit 221 and the fourth intermittence unit 222 of the second intermittence mechanism 220, so that the motor M and the spindle unit SP are in the power transmission state via the first transmission mechanism T1.

An external force in a bending direction input from the spindle unit SP is transmitted to the rotary damper 250 via the first transmission mechanism T1. Specifically, an input shaft 251 of the rotary damper 250 is provided with an input gear 252 that meshes with the first drive gear 183 of the first transmission mechanism T1. A one-way clutch 253 is provided between the input shaft 251 and the input gear 252 to transmit the rotation of the first transmission mechanism T1 in one direction to the rotary damper 250 and cut off the rotation in the opposite direction. As a result, even in the first transmission state, when the motor M is driven in power running, it is possible to cut off the power transmission to the rotary damper 250, and when the motor M is not driven in power running (under zero torque control or regeneration control), it is possible to transmit the external force input from the spindle unit SP to the rotary damper 250 and attenuate the external force.

In the electric prosthetic leg 1 configured in this way, it is possible to smoothly perform a step ascending operation, which has been required to be done one by one by a leg on a non-prosthetic leg side, with a passive prosthetic leg including a passive damper in the related art.

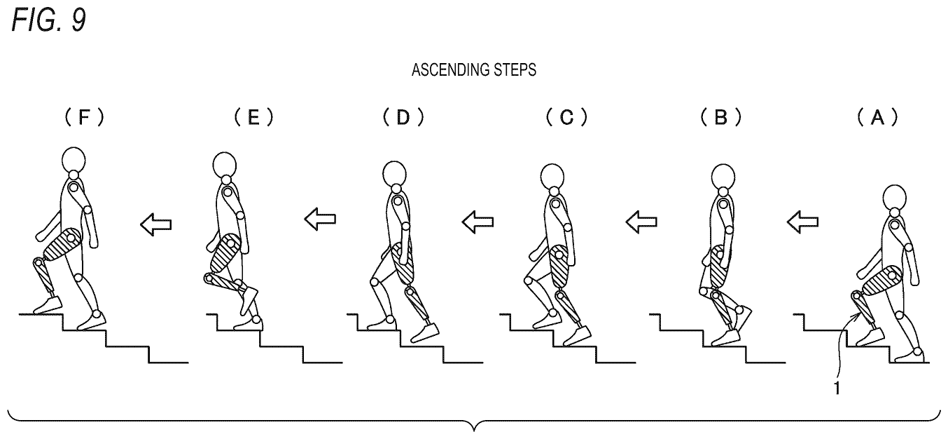

Specifically, as shown in (A) to (B) of FIG. 9, large power is required when the knee joint mechanism 130 is stretched from a bent state, in a state in which a load is applied to the electric prosthetic leg 1 when moving the electric prosthetic leg 1 forward to ascending steps.

In this case, the transmission T enters the second transmission state in which the first operating rod 231 and the second operating rod 241 are in the lower position. In the second transmission state, the large diameter portions 231c and 241c of the first operating rod 231 and the second operating rod 241 forcefully free the third intermittence unit 221 and the fourth intermittence unit 222 of the second intermittence mechanism 220, so that the motor M and the spindle unit SP are in the power transmission state via the first transmission mechanism T1.

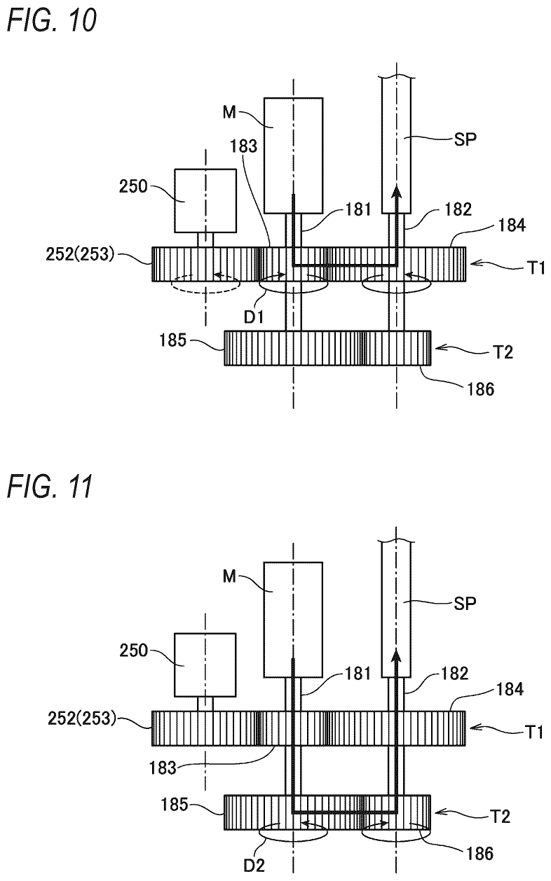

In this state, when the motor M is rotated in a first direction (direction D in FIG. 10), the power of the motor M is transmitted to the first shaft 181, the first intermittence unit 211 of the first intermittence mechanism 210, the first drive gear 183, the first driven gear 184, the second intermittence unit 212 of the first intermittence mechanism 210, the second shaft 182, and the spindle unit SP. As a result, the sleeve 174 is translated away from the transmission T, and the length of the spindle unit SP of the expansion-contraction device 140 is increased (extended). At the same time, the above-knee member 120, to which the sleeve 174 is attached, rotates around the pivoting portion 135 with respect to the below-knee member 110, to which the transmission T is attached, so that the second formed angle θ2 becomes larger and the first formed angle θ1 becomes smaller. As a result, the knee joint mechanism 130 stretches. Since the power for this stretching is power whose torque is increased during deceleration by the first transmission mechanism T1, even when a large load is applied to the electric prosthetic leg 1 when moving the electric prosthetic leg 1 forward to ascending steps, it is also possible to reliably stretch the knee joint mechanism 130 from the bent state.

On the other hand, in order to smoothly ascend steps, as shown in (D) to (E) of FIG. 9, it is necessary to bend (lift) the knee joint mechanism 130 from the stretched state while a load is applied to the healthy leg. When the knee joint mechanism 130 is bent from the stretched state, large power is not required, but quick action is required.

In this case, the transmission T enters the first transmission state in which the first operating rod 231 and the second operating rod 241 are in the upper position. In the first transmission state, the large diameter portions 231c and 241c of the first operating rod 231 and the second operating rod 241 forcefully free the first intermittence unit 211 and the second intermittence unit 212 of the first intermittence mechanism 210, so that the motor M and the spindle unit SP are in the power transmission state via the second transmission mechanism T2.

In this state, when the motor M is rotated in a second direction (direction D2 in FIG. 11), the power of the motor M is transmitted to the first shaft 181, the third intermittence unit 221 of the second intermittence mechanism 220, the second drive gear 185, the second driven gear 186, the fourth intermittence unit 222 of the second intermittence mechanism 220, the second shaft 182, and the spindle unit SR. As a result, the sleeve 174 is translated to approach the transmission T, and the length of the spindle unit SP of the expansion-contraction device 140 is decreased (contracted). At the same time, the below-knee member 110, to which the transmission T is attached, rotates around the pivoting portion 135 with respect to the above-knee member 120, to which the sleeve 174 is attached, so that the second formed angle θ2 becomes smaller and the first formed angle θ1 becomes larger. As a result, the knee joint mechanism 130 is bent. Since the power for this bending is power whose torque is reduced in acceleration by the second transmission mechanism T2, it becomes possible to quickly bend the knee joint mechanism 130.

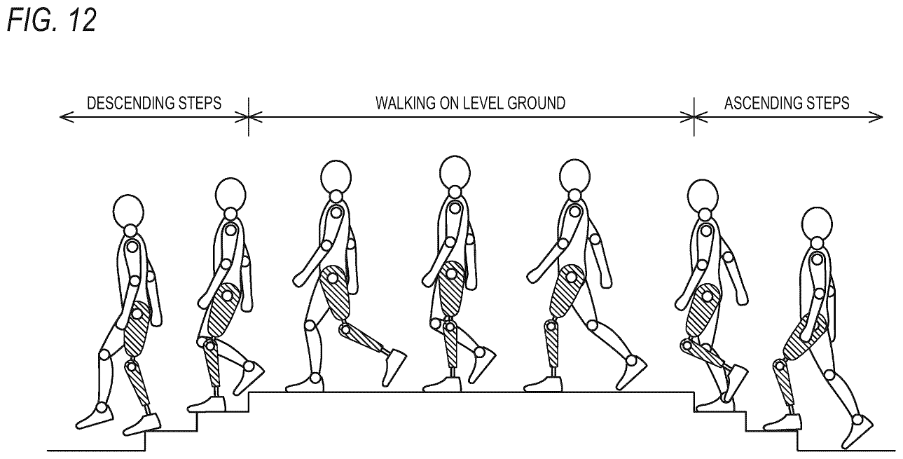

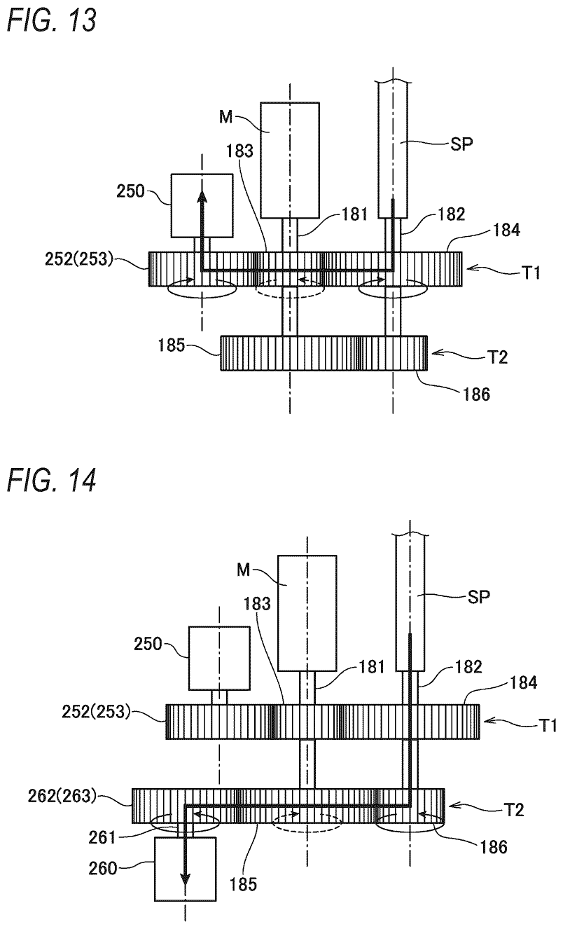

When descending steps and walking on level ground as shown in FIG. 12, as shown in FIG. 13, by attenuating the external force in the bending direction input from the spindle unit SP by the rotary damper 250, the knee joint mechanism 130 can be smoothly bent.

In this case, the transmission T enters the second transmission state in which the first operating rod 231 and the second operating rod 241 are in the lower position. In the second transmission state, the large diameter portions 231c and 241c of the first operating rod 231 and the second operating rod 241 forcefully free the third intermittence unit 221 and the fourth intermittence unit 222 of the second intermittence mechanism 220, so that the motor M and the spindle unit SP are in the power transmission state via the first transmission mechanism T1.

In this state, when the motor M is under zero torque control, the external force in the bending direction input from the spindle unit SP is transmitted to the second shaft 182, the second intermittence unit 212 of the first intermittence mechanism 210, the first driven gear 184, the first drive gear 183, the input gear 252, the one-way clutch 253, and the rotary damper 250. As a result, the external force in the bending direction input from the spindle unit SP is attenuated by the rotary damper 250, allowing the knee joint mechanism 130 to bend smoothly. Note that the motor M may under regeneration control instead of the zero torque control. In this way, the attenuation performance during bending can be enhanced.

Next, a modification of the electric prosthetic leg 1 according to the first embodiment will be described with reference to FIG. 14. Here, the same reference numerals as in the above embodiment are used for the sane configurations as in the above embodiment, and the description of the above embodiment may be incorporated.

As shown in FIG. 14, the electric prosthetic leg 1 according to this modification is different from the above embodiment in that a second rotary damper 260 for attenuating an external force in a stretching direction input from the spindle unit SP during walking on level ground is provided.

The external force in the stretching direction input from the spindle unit SP is transmitted to the second rotary damper 260 via the second transmission mechanism T2. Specifically, an input shaft 261 of the second rotary damper 260 is provided with an input gear 262 that meshes with the second drive gear 185 of the second transmission mechanism T2. A one-way clutch 263 is provided between the input shaft 261 and the input gear 262 to transmit the rotation of the second transmission mechanism T2 in one direction to the second rotary damper 260 and cut off the rotation in the opposite direction. As a result, even in the first transmission state, when the motor M is driven in power running, it is possible to cut off the power transmission to the second rotary damper 260, and when the motor M is not driven in power running (under zero torque control or regeneration control), it is possible to transmit the external force in the stretching direction input from the spindle unit SP to the second rotary damper 260 and attenuate the external force.

Specifically, when the external force in the stretching direction input from the spindle unit SP is attenuated by the second rotary damper 260, the transmission T is in the first transmission state in which the first operating rod 231 and the second operating rod 241 are in the upper position. In the first transmission state, the large diameter portions 231c and 241c of the first operating rod 231 and the second operating rod 241 forcefully free the first intermittence unit 211 and the second intermittence unit 212 of the first intermittence mechanism 210, so that the motor M and the spindle unit SP are in the power transmission state via the second transmission mechanism T2.

In this state, when the motor M is under zero torque control, the external force in the stretching direction input from the spindle unit SP is transmitted to the second shaft 182, the fourth intermittence unit 222 of the second intermittence mechanism 220, the second driven gear 186, the second drive gear 185, the input gear 262, the one-way clutch 263, and the second rotary damper 260. As a result, the external force in the stretching direction input from the spindle unit SP is attenuated by the second rotary damper 260, allowing the knee joint mechanism 130 to progress smoothly. Note that the motor M may under regeneration control instead of the zero torque control. In this way, the attenuation performance during stretching can be enhanced.

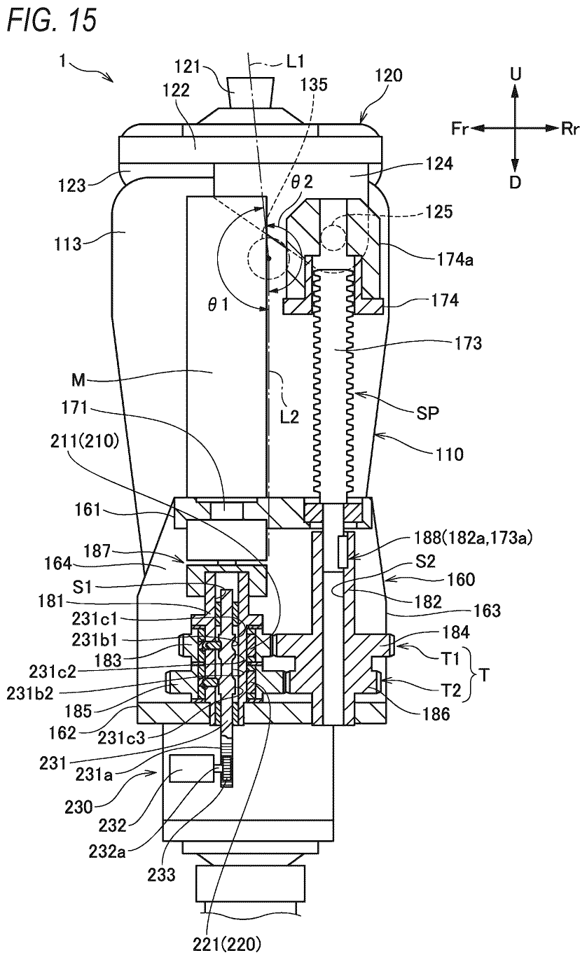

Next, the electric prosthetic leg 1 according to a second embodiment and a third embodiment of the present invention will be described with reference to FIGS. 15 to 26. However, descriptions of configurations common to those in the first embodiment will be omitted, or the description of the first embodiment may be incorporated by using the same reference numerals as in the first embodiment.

The transmission T according to the first embodiment is different from the transmission T according to the second embodiment and the third embodiment in that the transmission T according to the first embodiment includes four two-way clutches (intermittence units 211, 212, 221, 222) configured by combining two one-way clutches 270 with a forced free function, and these two-way clutches are turned on and off by two actuators (servomotors 232 and 242), whereas the transmission T according to the second embodiment and the third embodiment includes two two-way clutches with a forced free function, and these two-way clutches are turned on and off by one actuator. According to the second embodiment and the third embodiment, the number of parts of the transmission T can be reduced, and the structure thereof can be simplified and the cost can be reduced. Hereinafter, the configuration of the transmission T of the second embodiment and the third embodiment, and the configuration and operation of the two-way clutch of the second embodiment and the third embodiment will be sequentially explained.

As shown in FIG. 15, same as the transmission T according to the first embodiment, the transmission T according to the second embodiment includes the first transmission mechanism T1 that transmits the power of the motor M to the spindle unit SP at the first transmission ratio, and the second transmission mechanism T2 that transmits the power of the motor M to the spindle unit SP at the second transmission ratio, which is different from the first transmission ratio. The first transmission mechanism T1 is switched between a power cutoff state and a power connection state by the first intermittence mechanism 210, and the second transmission mechanism T2 is switched between a power cutoff state and a power connection state by the second intermittence mechanism 220.

The first transmission mechanism T1 of the second embodiment includes the first shaft 181 mechanically connected to the output shaft 171 of the motor M, the second shaft 182 mechanically connected to the spindle 173 of the spindle unit SP, the first drive gear 183 that is relatively rotatably provided on the first shaft 181, and the first driven gear 184 that is integrally rotatably provided on the second shaft 182 and rotates in synchronization with the first drive gear 183.

The second transmission mechanism T2 of the second embodiment includes the first shaft 181, the second shaft 182, the second drive gear 185 that is relatively rotatably provided on the first shaft 181, and the second driven gear 186 that is integrally rotatably provided on the second shaft 182 and rotates in synchronization with the second drive gear 185.

In the second embodiment, the first intermittence mechanism 210 includes the first intermittence unit 211 provided between the first drive gear 183 and the first shaft 181, and the second intermittence mechanism 220 includes the third intermittence unit 221 provided between the second drive gear 185 and the first shaft 181. That is, in the transmission T of the second embodiment, the intermittence units 211 and 221 are provided between the first shaft 181 and the gears 183 and 185, and the intermittence units 212 and 222 are not provided between the second shaft 182 and the gears 184 and 186.

These intermittence units 211 and 221 have a common configuration, and are configured to be switchable between the cutoff state in which power transmission is cut off, and the power transmittable state in which rotational power in both one direction and the other direction can be transmitted. Details thereof will be described later.

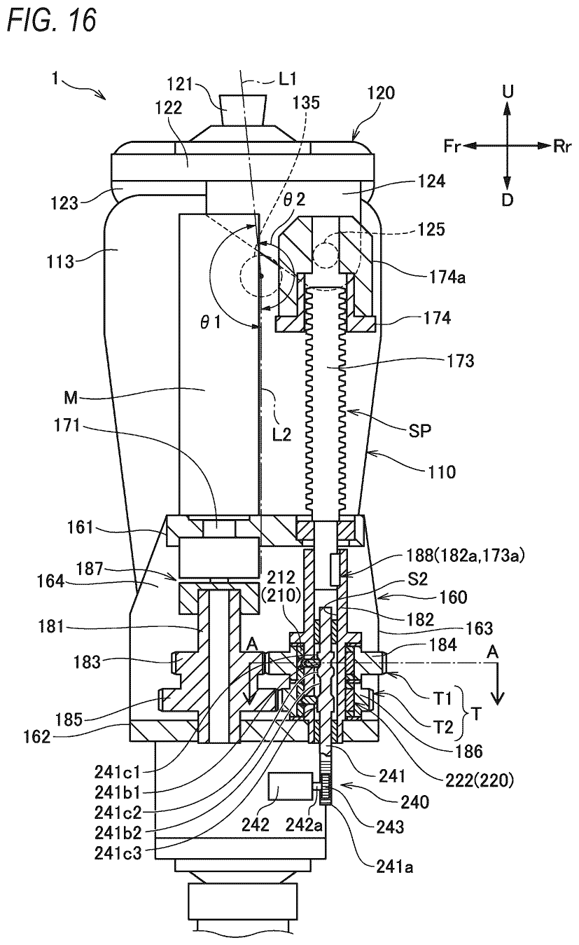

As shown in FIG. 16, same as the transmission T according to the second embodiment, the transmission T according to the third embodiment includes the first transmission mechanism T1, the second transmission mechanism T2, the first intermittence mechanism 210, and the second intermittence mechanism 220.

The first transmission mechanism T1 of the third embodiment includes the first shaft 181, the second shaft 182, the first drive gear 183 that is integrally rotatably provided on the first shaft 181, and the first driven gear 184 that is relatively rotatably provided on the second shaft 182 and rotates in synchronization with the first drive gear 183.

The second transmission mechanism T2 of the third embodiment includes the first shaft 181, the second shaft 182, the second drive gear 185 that is integrally rotatably provided on the first shaft 181, and the second driven gear 186 that is relatively rotatably provided on the second shaft 182 and rotates in synchronization with the second drive gear 185.

In the third embodiment, the first intermittence mechanism 210 includes the second intermittence unit 212 provided between the first driven gear 184 and the second shaft 182, and the second intermittence mechanism 220 includes the fourth intermittence unit 222 provided between the second driven gear 186 and the second shaft 182. That is, in the transmission T of the third embodiment, the intermittence units 212 and 222 are provided between the second shaft 182 and the gears 184 and 186, and the intermittence units 211 and 221 are not provided between the first shaft 181 and the gears 183 and 185.

These intermittence units 212 and 222 have a common configuration, and are configured to be switchable between the cutoff state in which power transmission is cut off, and the power transmittable state in which rotational power in both one direction and the other direction can be transmitted.

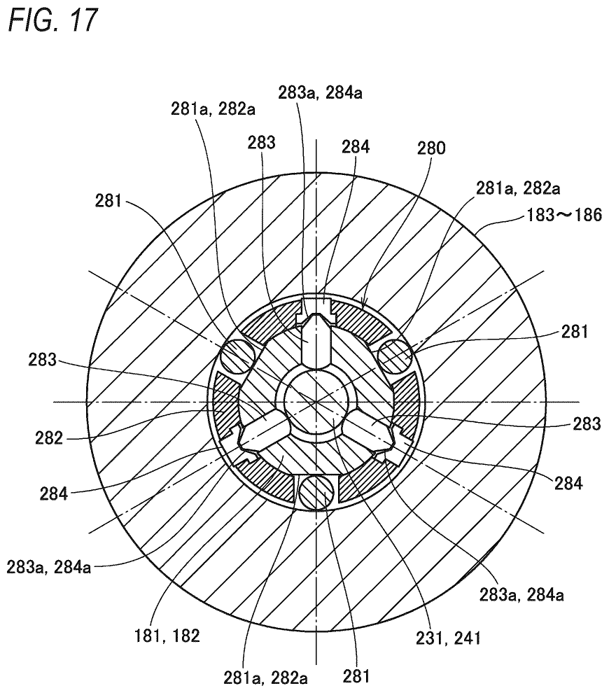

The intermittence units 211 and 221 of the second embodiment and the intermittence units 212 and 222 of the third embodiment are configured using the two-way clutch 280 with a forced free function, as shown in FIG. 17. The two-way clutch 280 includes the plurality of (three in these embodiments) rollers 281 arranged between the outer peripheral surface portions of the shafts 181 and 182 and the inner peripheral surface portions of the gears 183 to 186, the retainer 282 that holds the plurality of rollers 281 at predetermined intervals, the plurality of (three in these embodiment) pins 283 that penetrate the shafts 181 and 182 in the radial direction and are operated by the first operating mechanism 230 or the second operating mechanism 240 to the forced free position and the forced free release position, and the plurality of (three in these embodiment) guides 284 provided on the retainer 282 and defining a relative rotational position of the retainer 282 with respect to the shafts 181 and 182 when the pins 283 are in the forced free position.

A distance A in the radial direction (not shown) between the outer peripheral surface portions of the shafts 181 and 182 and the inner peripheral surface portions of the gears 183 to 186 is smaller than a diameter B (not shown) of the rollers 281. Flat portions 281a and 282a are formed on the outer peripheral portions of the shafts 181 and 182 at predetermined intervals in the circumferential direction, and on a center side in the circumferential direction of the flat portions 281a and 282a, the distance A is larger than the diameter B.

In other words, when the rollers 281 are held at center portions of the flat portions 281a and 282a in the circumferential direction, the rollers 281 do not mesh with the outer peripheral surface portions of the shafts 181 and 182 and the inner peripheral surface portions of the gears 183 to 186, and relative rotation between the shafts 181 and 182 and the gears 183 to 186 is allowed (forced free state).

On the other hand, when the rollers 281 are allowed to move in the circumferential direction relative to the shafts 181 and 182, the rollers 281 mesh with the outer peripheral surface portions of the shafts 181 and 182 and the inner peripheral surface portions of the gears 183 to 186, and the shafts 181 and 182 and the gears 183 to 186 are connected in a manner of being rotatable integrally in two directions (forced free release state).

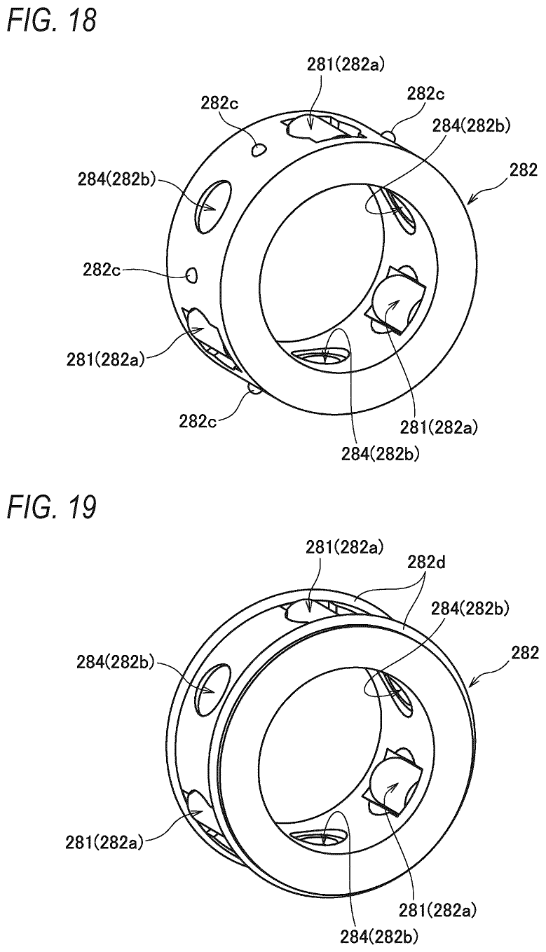

As shown in FIG. 18, the retainer 282 includes a plurality of roller holding portions 282a that are ring-shaped and capable of relative rotation with respect to the shafts 181, 182 and gears 183 to 186, and that hold the rollers 281, and a plurality of guide holding portions 282b that hold the guides 284.

A plurality of rubber balls 282c are embedded in an outer peripheral surface of the retainer 282 at predetermined intervals in the circumferential direction. These rubber balls 282c prevent unintended idling in the forced free release state by generating moderate friction between the gears 183 to 186 and the retainer 281. Note that a member that creates friction between the gears 183 to 186 and the retainer 282 may be an O-ring 282d as shown in FIG. 19. The rubber ball 282c and the O-ring 282d are effective in preventing idling, but can be omitted.

Returning to FIG. 17, each of the pins 283 includes a conical convex portion 283a on an outer end portion in the radial direction, and each of the guides 284 includes a conical concave portion 284a on an inner end portion in the radial direction that fits to (engages with) the convex portion 283a. When the convex portion 283a of the pin 283 fits to the concave portion 284a of the guide 284, the relative rotational position of the retainer 282 with respect to the shafts 181 and 182 is positioned at a predetermined position where the retainer 282 is in the forced free state by a guiding effect of the pins 283 and the guides 284.

As shown in FIGS. 15 and 16, the shafts 181, 182 include, in order from the top, first large diameter portions 231c1, 241c1, first small diameter portions 231b1, 241b1, second large diameter portions 231c2, 241c2, and second small diameter portions 231b2, 241b2 and third large diameter portions 231c, 241c3 formed with predetermined lengths and intervals. The shafts 181 and 182 are each provided so as to be able to control two intermittence units simultaneously, but may be provided separately for each intermittence unit.

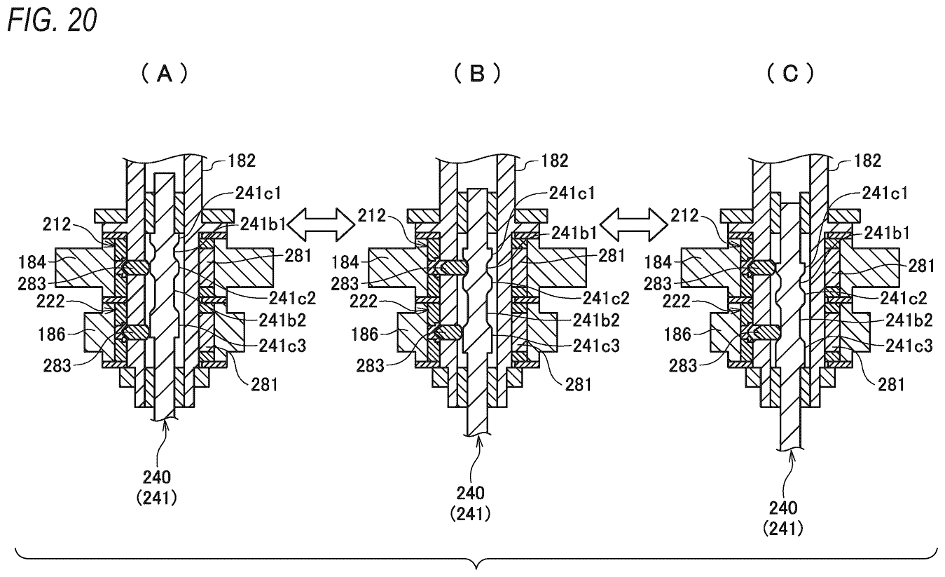

In the following description, the operation of the second operating mechanism 240 that simultaneously controls the intermittence units 212 and 222 of the third embodiment will be described with reference to FIG. 20.

As shown in FIG. 20, the intermittence units 212 and 222 are switched between the forced free state (hereinafter referred to as an OFF state as appropriate) and the forced free release state (hereinafter referred to as an ON state as appropriate) by the second operating mechanism 240.

When the second operating rod 241 of the second operating mechanism 240 is in an upper position shown in (A) of FIG. 20, the third large diameter portion 241c3 pushes out the pin 283 of the fourth intermittence unit 222 in an outer diameter direction while the second large diameter portion 241c2 pushes out the pin 283 of the second intermittence unit 212 in the outer diameter direction, so that the second intermittence unit 212 and the fourth intermittence unit 222 are in the off state.

When the second operating rod 241 of the second operating mechanism 240 is in a middle position shown in (B) of FIG. 20, the third large diameter portion 241c3 pushes out the pin 283 of the fourth intermittence unit 222 in the outer diameter direction while the first small diameter portion 241b1 allows the pin 283 of the second intermittence unit 212 to return in an inner diameter direction, so that the second intermittence unit 212 is in the on state and the fourth intermittence unit 222 is in the off state.

When the second operating rod 241 of the second operating mechanism 240 is in a lower position shown in (C) of FIG. 20, the second small diameter portion 241b2 allows the pin 283 of the fourth intermittence unit 222 to return in the inner diameter direction while the first large diameter portion 241c1 pushes out the pin 283 of the second intermittence unit 212 in the outer diameter direction, so that the second intermittence unit 212 is in the off state and the fourth intermittence unit 222 is in the on state.

Note that although illustration is omitted, the intermittence units 211 and 221 of the second embodiment are also switched between the forced free state and the forced free release state by the first operating mechanism 230. The first operating rod 231 of the first operating mechanism 230 is configured to be movable to the upper position (position corresponding to a position (A) in FIG. 20), the middle position (position corresponding to a position (B) in FIG. 20), and the lower position (position corresponding to a position (C) in FIG. 20). The first operating rod 231 of the first operating mechanism 230, in the upper position, pushes out the pin 283 of the first intermittence unit 211 and the third intermittence unit 221 in the outer diameter direction so that the first intermittence unit 211 and the third intermittence unit 221 are in the off state, and in the middle position, pushes out the pin 283 of the third intermittence unit 221 in the outer diameter direction while allowing the pin 283 of the first intermittence unit 211 to return in the inner diameter direction, so that the first intermittence unit 211 is in the on state and the third intermittence unit 221 is in the off state, and in the lower position, pushes out the pin 283 of the first intermittence unit 211 in the outer diameter direction while allowing the pin 283 of the third intermittence unit 221 to return in the inner diameter direction, so that the first intermittence unit 211 is in the off state and the third intermittence unit 221 is in the on state.

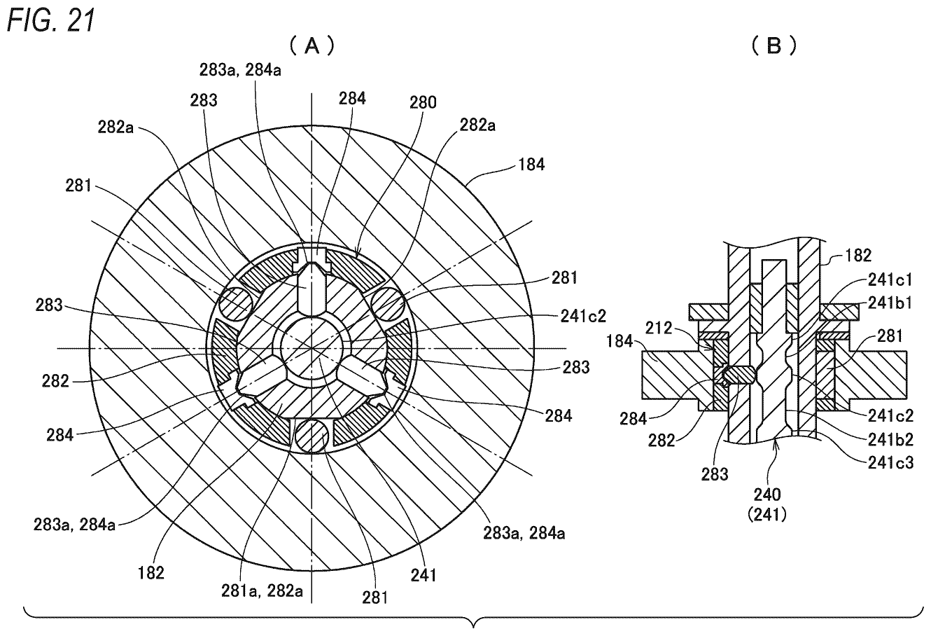

Next, the operation of the two-way clutch 280 will be described with reference to FIGS. 21 to 26, taking the second intermittence unit 212 of the third embodiment as an example. In the following example, the case of transition from (A) to (B) to (C) in FIG. 20 in the second intermittence unit 212 will be described as an example.

As shown in (A) and (B) of FIG. 21, when the second large diameter portion 241c2 of the second operating rod 241 pushes out the pin 283 of the second intermittence unit 212 in the outer diameter direction, the convex portion 283a of the pin 283 fits to the concave portion 284a of the guide 284, and the relative rotational position of the retainer 282 with respect to the second shaft 182 is fixed at a predetermined position. In this state, since the rollers 281 are held at a center portion in the circumferential direction of the flat portion 282a, the rollers 281 do not mesh with the outer peripheral surface portion of the second shaft 182 and the inner peripheral surface portion of the first driven gear 184, and the state becomes the off state in which the relative rotation between the second shaft 182 and the first driven gear 184 is allowed.

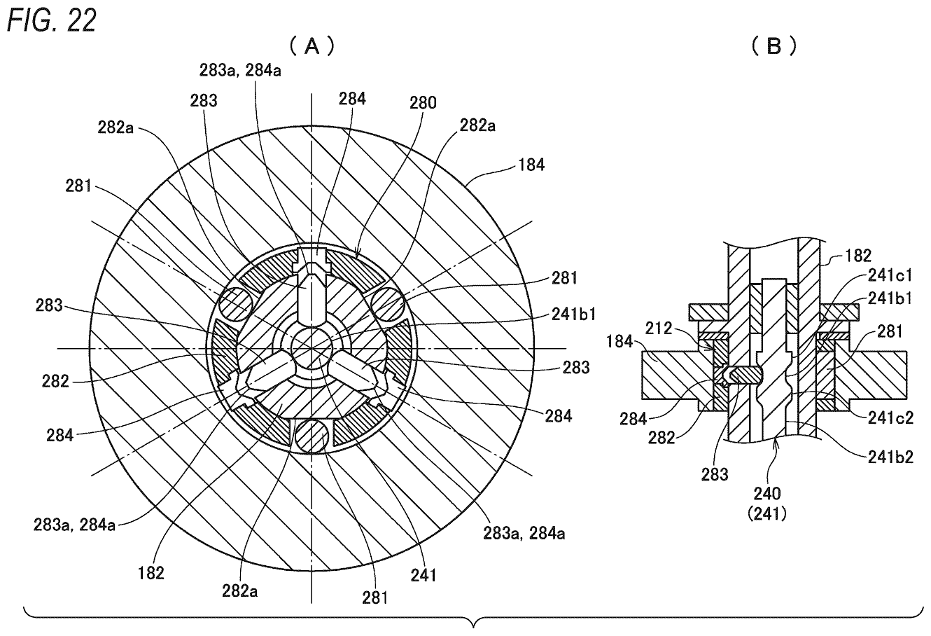

In FIG. 22, (A) and (B) show a state in which the second operating rod 241 moves from the position where the second large diameter portion 241c2 pushes out the pin 283 of the second intermittence unit 212 in the outer diameter direction to the position where the first small diameter portion 241b1 allows the pin 283 to return in the inner diameter direction. In FIG. 22, the pin 283 is moved in the inner diameter direction, but actually, at the timing when the relative rotation between the second shaft 182 and the first driven gear 184 occurs, the guides 284 of the retainer 282 that rotate together with the first driven gear 184 pushes the pin 283 back in the inner diameter direction on an inclined surface of the concave portion 284a.

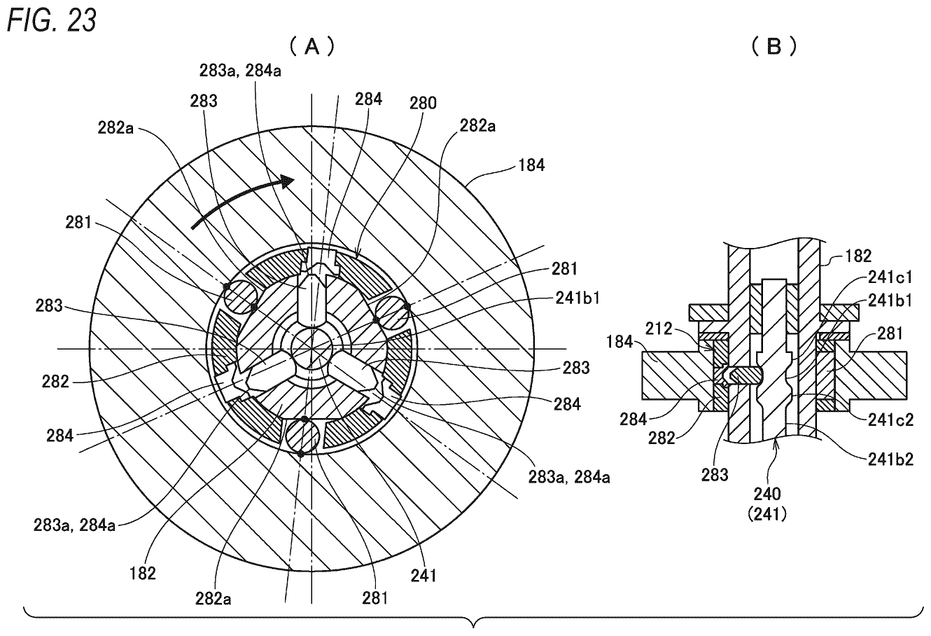

As shown in (A) and (B) of FIG. 23, in a state in which the pin 283 is allowed to return in the inner diameter direction, when relative rotation occurs between the second shaft 182 and the first driven gear 184 in a forward rotation direction shown by the arrow in the drawing, the retainer 282 that rotates together with the first driven gear 184 moves the roller 281 in the forward rotation direction with respect to the second shaft 182. As a result, the rollers 281 mesh with the outer peripheral surface portion of the second shaft 182 and the inner peripheral surface portion of the first driven gear 184, and a forward rotation on state is created in which the second shaft 182 and the first driven gear 184 are rotated integrally in the forward rotation direction.

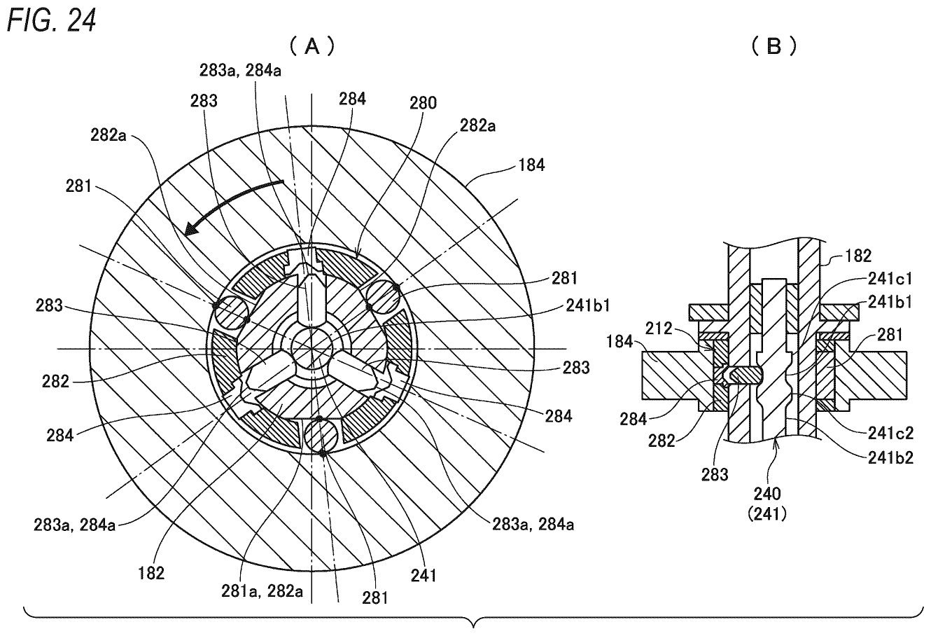

As shown in (A) and (B) of FIG. 24, in a state in which the pin 283 is allowed to return in the inner diameter direction, when relative rotation occurs between the second shaft 182 and the first driven gear 184 in a rearward rotation direction shown by the arrow in the drawing, the retainer 282 that rotates together with the first driven gear 184 moves the roller 281 in the rearward rotation direction with respect to the second shaft 182. As a result, the rollers 281 mesh with the outer peripheral surface portion of the second shaft 182 and the inner peripheral surface portion of the first driven gear 184, and a rearward rotation on state is created in which the second shaft 182 and the first driven gear 184 are rotated integrally in the rearward rotation direction.

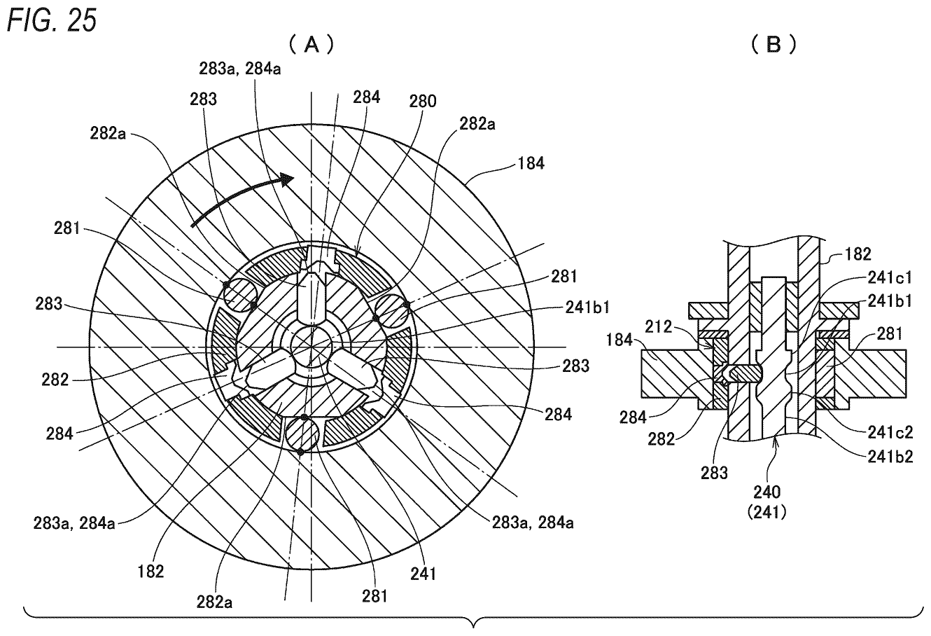

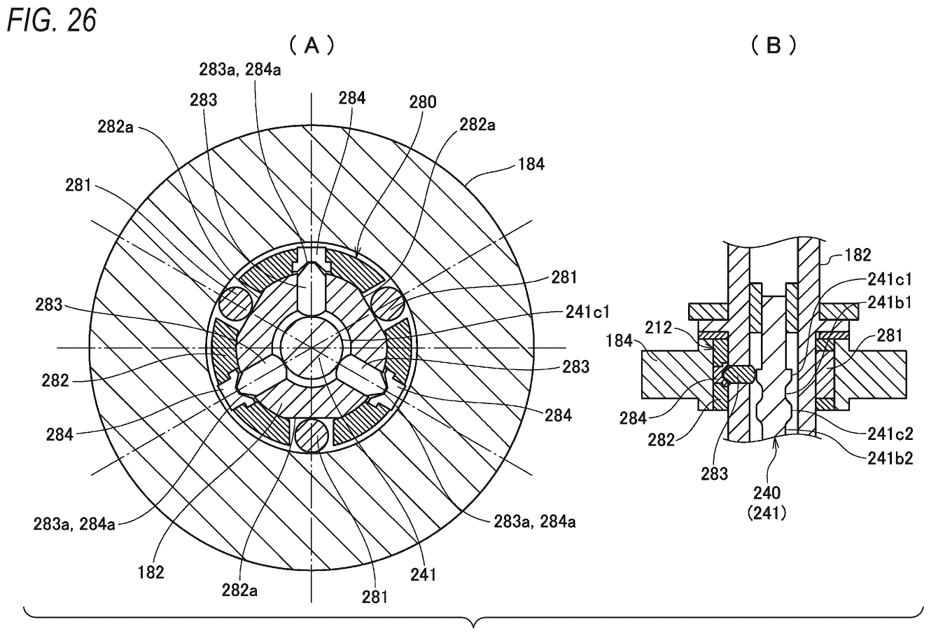

In the on state shown in (A) and (B) of FIG. 25, as shown in (A) and (B) of FIG. 26, when the second operating rod 241 moves from a position where the first small diameter portion 241b1 allows the pin 283 of the second intermittence unit 212 to return in the inner diameter direction, to a position where the first large diameter portion 241c1 pushes out the pin 283 in the outer diameter direction, the convex portion 283a of the pin 283 fits to the concave portion 284a of the guide 284, and due to the guiding effect of the pin 283 and the guide 284, the relative rotational position of the retainer 282 with respect to the second shaft 182 is fixed at a predetermined position. In this state, since the rollers 281 are held at a center portion in the circumferential direction of the flat portion 282a, the rollers 281 do not mesh with the outer peripheral surface portion of the second shaft 182 and the inner peripheral surface portion of the first driven gear 184, and the state becomes the off state in which the relative rotation between the second shaft 182 and the first driven gear 184 is allowed.

Although detailed description is omitted, the two-way clutch 280 of the fourth intermittence unit 222 of the third embodiment and the first intermittence unit 211 and the third intermittence unit 221 of the second embodiment operate in the same manner, and the two-way clutch 280 can take the off state, the forward rotation on state, and the rearward rotation on state. According to the transmission T according to the second embodiment and the third embodiment, the number of parts can be reduced, and the structure can be simplified and costs can be reduced as compared with the transmission T according to the first embodiment as described above. In the transmission T according to the third embodiment, when the motor M is on the upstream side and the spindle unit SP is on the downstream side in the power transmission path of the motor M, the second intermittence unit 212 and the fourth intermittence unit 222 are provided on the downstream side, and therefore, when each of the intermittence units 212 and 222 is in the off state, fewer rotating bodies are involved, and the operation of the electric prosthetic leg 1 becomes easy.

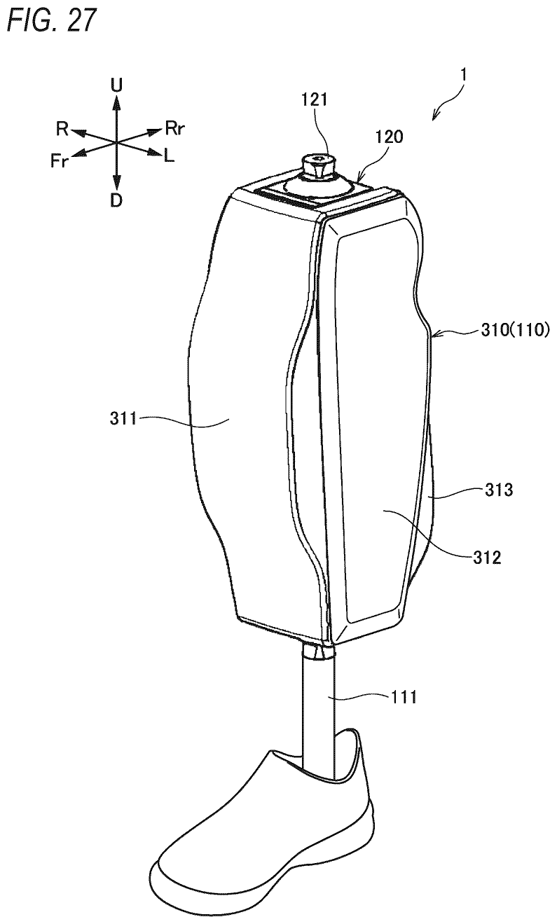

Next, the electric prosthetic leg 1 according to a fourth embodiment of the present invention will be described with reference to FIGS. 27 to 32. Here, the sane reference numerals as in the second embodiment are used for the same configurations as in the second embodiment, and the description of the above embodiment may be incorporated.

The electric prosthetic leg 1 according to the fourth embodiment is mainly different from that according to the third embodiment in a housing configuration, disposition of the spindle unit SP, the sleeve 174 of the spindle unit SP being coupled to the above-knee member 120 via a link member 320, arrangement of the first transmission mechanism T1 and the second transmission mechanism T2, shapes of drive gears 183, 185 and the driven gears 184, 186, and a stretching assist mechanism 330 being provided for assisting the stretching of the knee joint mechanism 130 using a force accumulated when the knee is bent. Details of each difference will be described below.

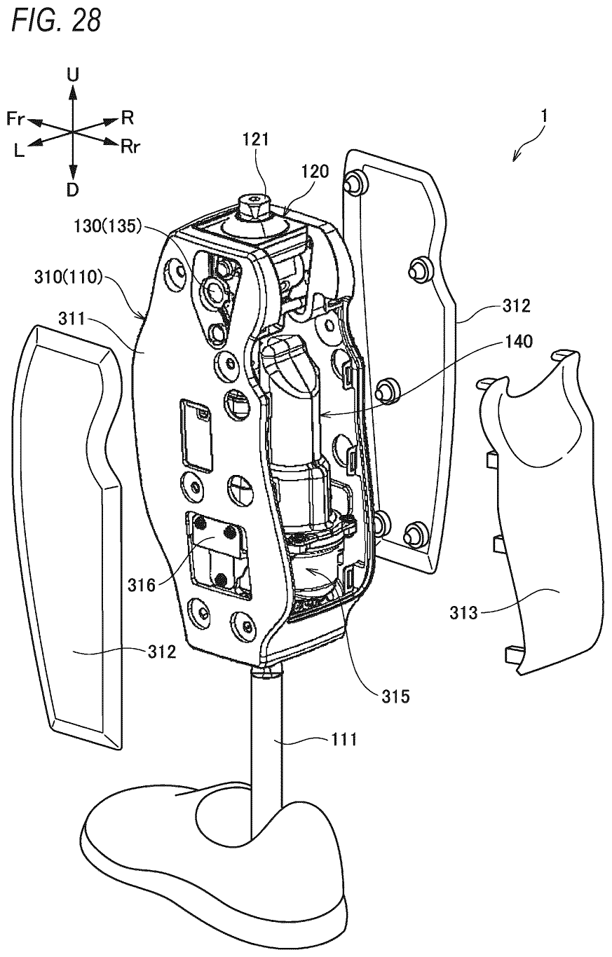

As shown in FIGS. 27 and 28, a housing 310 of the electric prosthetic leg 1 according to the fourth embodiment is open at an upper portion and a rear portion, and includes a box-shaped main frame 311 constituting the below-knee member 110, a side cover 312 that covers both left and right side surfaces of the main frame 311, and a detachable rear cover 313 that covers the rear opening of the main frame 311 in an openable and closable manner.

The above-knee member 120 is provided on an upper portion of the main frame 311 via the pivoting portion 135, and the leg portion 111 is provided on a lower portion of the main frame 311. The unitized expansion-contraction device 140 is incorporated inside the main frame 311. The expansion-contraction device 140 extends in the upper-lower direction, and is mechanically connected to the above-knee member 120 on one side in the extending direction and mechanically connected to the below-knee member 110 on the other side in the extending direction. Note that the term “mechanically connected” is a concept that includes a configuration of direct connection and a configuration of connection via another member. More specifically, in the expansion-contraction device 140, an upper end portion of a link member 320 (described below) positioned on one side in the extending direction is mechanically connected to the above-knee member 120 at a second pivoting portion 322, and a unit case 315 positioned on the other side in the extending direction is mechanically connected to the main frame 311, that is, the below-knee member 110 via a bracket 316.

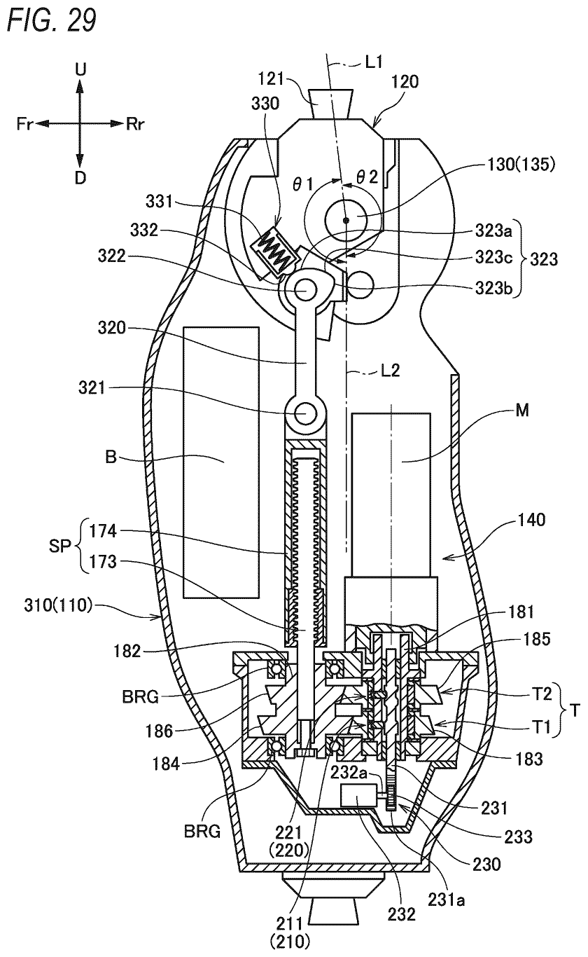

As shown in FIG. 29, the transmission T includes the first transmission mechanism T1, the second transmission mechanism T2, the first intermittence mechanism 210, and the second intermittence mechanism 220. Note that the transmission T according to the fourth embodiment differs from the transmission T according to the second embodiment and the third embodiment in that the first transmission mechanism T1 is disposed below the second transmission mechanism T2.

The first transmission mechanism T1 includes the first shaft 181 mechanically connected to the output shaft of the motor M, the second shaft 182 mechanically connected to the spindle 173 of the spindle unit SP, the first drive gear 183 that is relatively rotatably provided on the first shaft 181, and the first driven gear 184 that is integrally rotatably provided on the second shaft 182 and rotates in synchronization with the first drive gear 183.

The second transmission mechanism T2 includes the first shaft 181, the second shaft 182, the second drive gear 185 that is relatively rotatably provided on the first shaft 181, and the second driven gear 186 that is integrally rotatably provided on the second shaft 182 and rotates in synchronization with the second drive gear 185.

The first intermittence mechanism 210 includes the first intermittence unit 211 provided between the first drive gear 183 and the first shaft 181, and the second intermittence mechanism 220 includes the third intermittence unit 221 provided between the second drive gear 185 and the first shaft 181. That is, in the transmission T of the fourth embodiment, the intermittence units 211 and 221 are provided between the first shaft 181 and the gears 183 and 185, and the intermittence units 212 and 222 are not provided between the second shaft 182 and the gears 184 and 186. Note that the fourth embodiment is similar to the second embodiment in that each intermittence unit 211, 221 includes the two-way clutch 280, and therefore, detailed description will be omitted. Even in the present embodiment, when the spindle 173 rotates to one side by receiving the rotational power of the motor M transmitted by the transmission T, the sleeve 174 is translated away from the transmission T, and when the spindle 173 rotates to the other side, the sleeve 174 is translated in a direction of approaching the transmission T. Note that the translational movement of the sleeve 174 away from the transmission T may be referred to as an expanding operation of the spindle unit SP, and conversely, the translational movement of the sleeve 174 approaching the transmission T may be referred to as a contracting operation of the spindle unit SP.

As shown in FIGS. 29 to 32, in the electric prosthetic leg 1 according to the fourth embodiment, the spindle unit SP is disposed in front of the pivoting portion 135, and the knee joint mechanism 130 is bent in response to the expanding operation of the spindle unit SP, and the knee joint mechanism 130 is stretched in response to the contracting operation of the spindle unit SF.

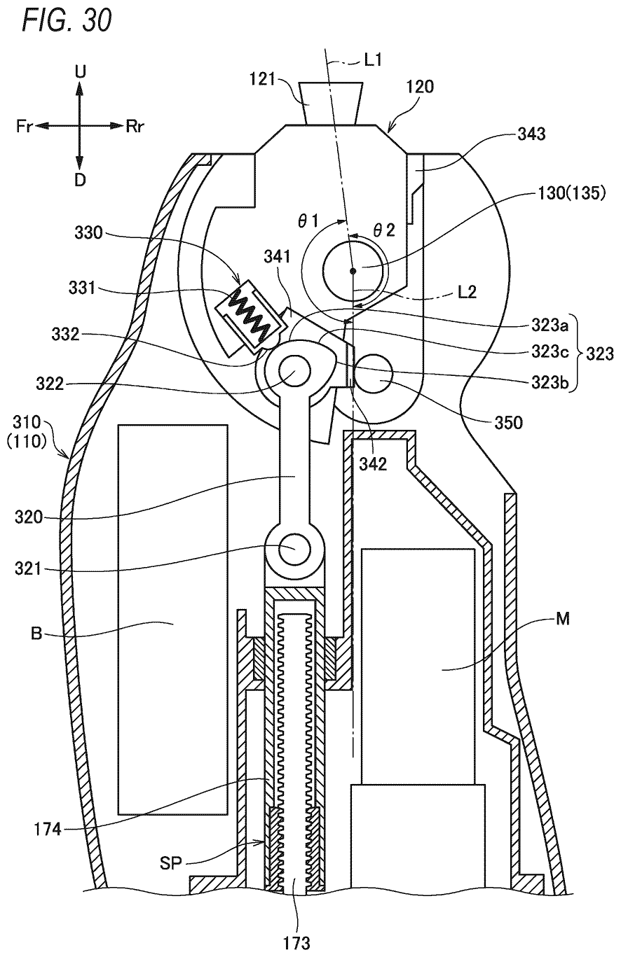

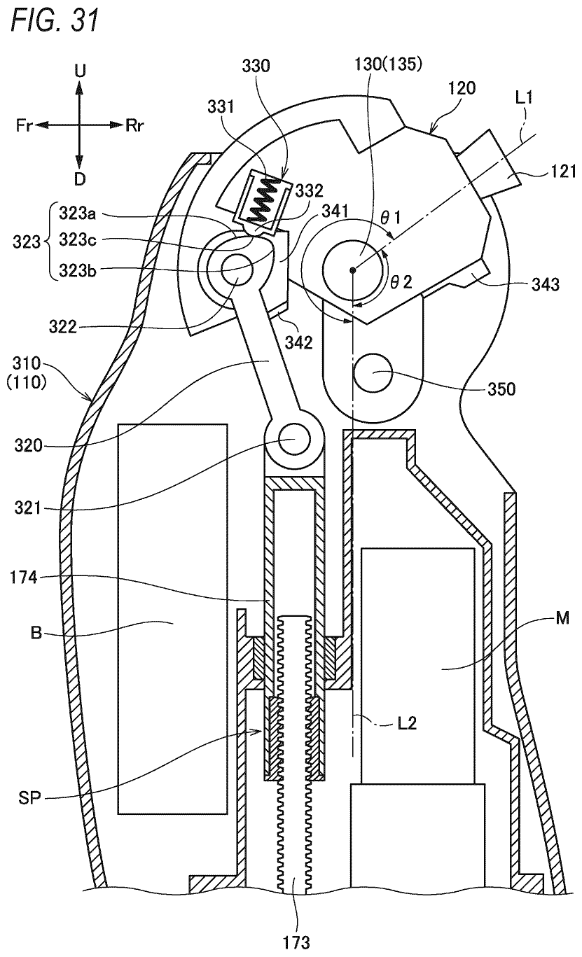

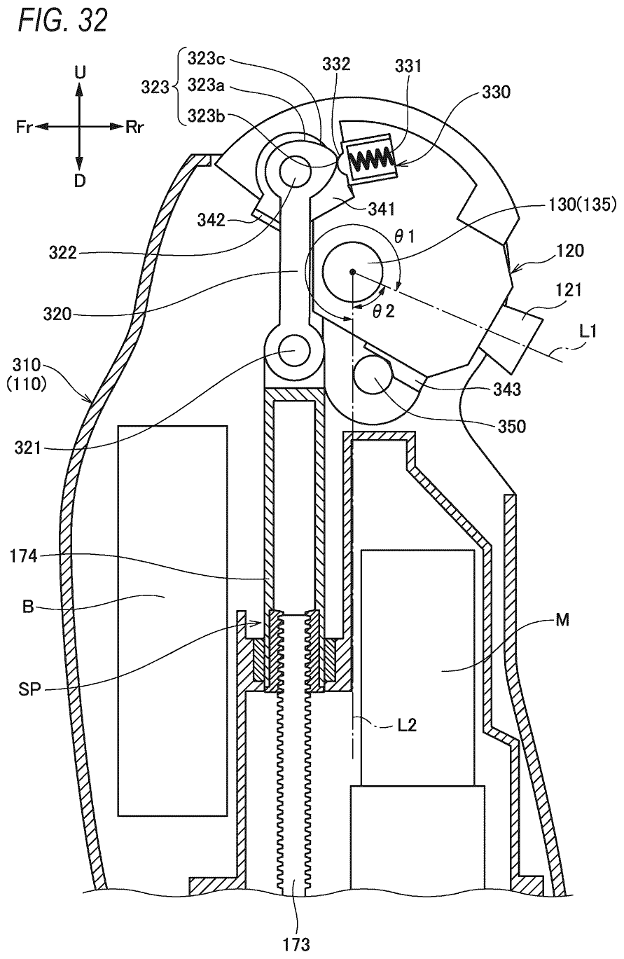

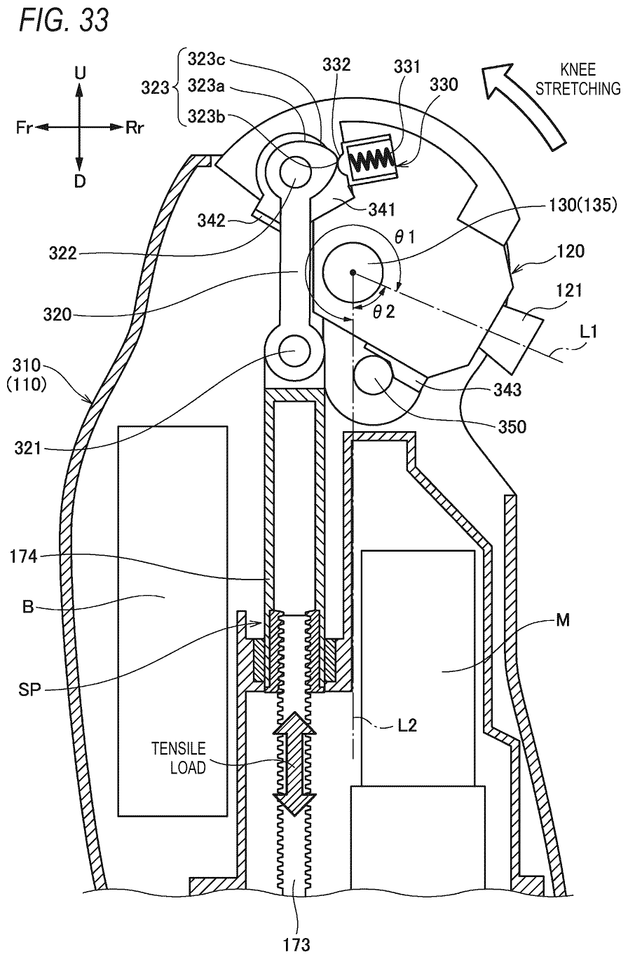

FIG. 30 shows a stretched state of the knee joint mechanism 130, in which the first formed angle θ1 is approximately 170°, and the second formed angle θ2 is approximately 190°. FIG. 31 shows a bending state of the knee joint mechanism 130, in which the first formed angle θ1 is approximately 260°, and the second formed angle θ2 is approximately 120°. FIG. 32 is a diagram showing a maximum bent state of the knee joint mechanism 130, in which the first formed angle θ1 is approximately 310°, and the second formed angle θ2 is approximately 50°. FIG. 33 is a diagram showing the maximum bent state of the electric prosthetic leg 1 according to the fourth embodiment, and illustrating angles formed between the above-knee member 120 and the below-knee member 110, and operation and load on the spindle unit SP.

Same as the first to third embodiments, the angles formed between the above-knee member 120 and the below-knee member 110 are angles defined by the first virtual line L1 connecting the center of the pivoting portion 135 of the knee joint mechanism 130 and the adapter 121 of the above-knee member 120 and the second virtual line L2 that extends downward in the vertical direction through the center of the pivoting portion 135 of the knee joint mechanism 130 and the below-knee member 110. One side of one circumference centering on the pivoting portion 135 of the knee joint mechanism 130 and having angles formed between the below-knee member 110 and the above-knee member 120 is defined as the first formed angle θ1, and the other side of the one circumference is defined as the second formed angle θ2. When smaller one of minimum formed angles, of the first formed angle θ1 and the second formed angle θ2, formed in a range of relative movement between the below-knee member 110 and the above-knee member 120 is defined as the second formed angle θ2, the angle formed on a back side of the knee of the user of the electric prosthetic leg 1 is defined as the second formed angle θ2. The first formed angle θ1 takes a value of approximately 170° to 310°, and the second formed angle θ2 takes a value of approximately 50° to 190°.

In the electric prosthetic leg 1 according to the present embodiment, at least a part of the expansion-contraction device 140 is disposed on the first formed angle θ1 side (shin side) with respect to the second virtual line L2. More specifically, the spindle unit SP of the expansion-contraction device 140 is provided on the first formed angle θ1 side (shin side) with respect to the second virtual line L2. That is, the spindle unit SP of the first to third embodiments is provided on the second formed angle θ2 side (calf side) with respect to the second virtual line L2, whereas the spindle unit SP of the fourth embodiment is provided on the first formed angle θ1 side (shin side) with respect to the second virtual line L2. Therefore, when the second formed angle θ2 becomes smaller and the first formed angle θ1 becomes larger, the length of the spindle unit SP is increased and the knee joint mechanism 130 is bent. On the other hand, when the second formed angle θ2 becomes larger and the first formed angle θ1 becomes smaller, the length of the spindle unit SP is decreased and the knee joint mechanism 130 is stretched. In other words, the expansion-contraction device 140 is provided so that the length of the spindle unit SP decreases (becomes smaller) when the second formed angle θ2 becomes larger.

On the other hand, in the present embodiment, the motor M of the expansion-contraction device 140 is provided on the second formed angle θ2 side (calf side) with respect to the second virtual line L2.

According to such a configuration, as shown by the outlined arrow in FIG. 33, when the spindle 173 rotates to the other side during a high torque operation of stretching the knee joint mechanism 130 from the bent state ((A) to (B) of FIG. 9), the sleeve 174 translates to approach the transmission T, and a force of pulling up from the sleeve 174 acts on the spindle 173, and a tensile load (tensile force) is generated on the spindle 173 in a direction of gravity shown by the hatched arrow in FIG. 33. Therefore, during the high torque operation of stretching the knee joint mechanism 130 from the bent state, since a pulling force from the sleeve 174 side acts on the spindle 173 of the spindle unit SP and a tensile load is generated, buckling deformation of the spindle 173 can be prevented. Since the spindle 173 is formed from a material (for example, metal) that is stronger in tension than in compression, it is more durable than one that is subjected to compressive forces during this operation. Note that the case where the knee joint mechanism 130 is stretched from the bent state equals to the case where the second formed angle θ2 becomes larger and the first formed angle θ1 becomes smaller. Therefore, it can be said that the expansion-contraction device 140 of the present embodiment is provided so that a tensile force acts on the spindle 173 of the spindle unit SP when the second formed angle θ2 becomes larger.

To describe a support structure that supports the spindle 173, as shown in FIG. 29, the spindle 173 is integrally coupled with the second shaft 182 that is integrated with the first driven gear 184 and the second driven gear 186, and the second shaft 182 is supported by the unit case 315 via a pair of upper and lower bearings BRG. When the second formed angle θ2 between the above-knee member 120 and the below-knee member 110 becomes larger and the first formed angle θ1 becomes smaller in response to the contracting operation of the spindle unit SF, since a pulling force acts on the spindle 173 from the sleeve 174 in a direction opposite to the direction of gravity, the load applied to the bearings BRG is reduced. Therefore, it is possible to prevent the bearings BRG from increasing in size.

The gears 183 to 186 are all helical gears, and when the motor M is driven in power running, a thrust force is applied from the drive gears 183 and 185 to the driven gears 184 and 186. By configuring the gears 183 to 186 so that this thrust force acts on the spindle 173 in a direction opposite to the direction of gravity, it becomes possible to avoid increasing the size of the support structure that supports the spindle 173.

As shown in FIG. 29, in the electric prosthetic leg 1 of the fourth embodiment, the sleeve 174 of the spindle unit SP is coupled with the above-knee member 120 via the link member 320. Specifically, the upper end portion of the sleeve 174 is coupled with the lower end portion of the link member 320 via a first pivoting portion 321, and the upper end portion of the link member 320 is coupled with the above-knee member 120 via the second pivoting portion 322. In this way, the knee joint mechanism 130 can be bent and stretched in accordance with the expansion and contraction of the spindle unit SP, without supporting the entire expansion-contraction device 140 in a swingable manner as in the electric prosthetic leg 1 according to the first to third embodiments.

As described above, FIG. 30 shows a stretched state of the electric prosthetic leg 1 according to the fourth embodiment, and FIG. 31 shows a stretching state of the electric prosthetic leg 1, and FIG. 32 shows the maximum bent state of the electric prosthetic leg 1. Note that in walking with the electric prosthetic leg 1, the maximum bent state shown in FIG. 32 does not occur. In FIG. 30, a first stopper 342 attached to a support piece 341 that supports the second pivoting portion 322 comes into contact with a position regulating pin 350, and the knee joint mechanism 130 is prevented from bending in the opposite direction. In FIG. 32, a second stopper 343 attached to the above-knee member 120 comes into contact with the position regulating pin 350, and the knee joint mechanism 130 is prevented from further bending from the maximum bent state. Note that in FIGS. 30 to 32, the symbol B represents a battery that supplies electric power to the motor M.

As shown in FIG. 29, in the electric prosthetic leg 1 according to the fourth embodiment, the battery B is provided on the first formed angle θ1 side (shin side) with respect to the second virtual line L2. That is, the battery B is provided together with the spindle unit SP on the first formed angle θ1 side (shin side) with respect to the second virtual line L2. On the other hand, the motor M is provided on the second formed angle θ2 side (calf side) with respect to the second virtual line L2. In other words, the battery B and the motor M are provided on opposite sides of the spindle unit SP.

As shown in FIGS. 29 to 32, the stretching assist mechanism 330 is provided between the upper end portion of the link member 320 and the above-knee member 120 to assist the stretching with the force accumulated when the knee joint mechanism 130 is bent. The stretching assist mechanism 330 includes a pressing portion 332 that presses the upper end portion of the link member 320 with a biasing force of a spring 331 (for example, a compression coil spring). A cam portion 323 is formed at the upper end portion of the link member 320. The cam portion 323 includes a small diameter outer peripheral portion 323a having a small diameter and centered on the second pivoting portion 322, a large diameter outer peripheral portion 323b with a long distance from the second pivoting portion 322, and a connecting outer peripheral portion 323c that connects the small diameter outer peripheral portion 323a and the large diameter outer peripheral portion 323b without any step.

As shown in FIG. 30, in the stretched state of the knee joint mechanism 130 with the spindle unit SP contracted, the pressing portion 332 is in contact with the small diameter outer peripheral portion 323a of the cam portion 323. As shown in FIG. 31, when the knee joint mechanism 130 is bent by expanding the spindle unit SP from the stretched state of the knee joint mechanism 130, a contact position between the pressing portion 332 and the cam portion 323 changes from the small diameter outer peripheral portion 323a to the large diameter outer peripheral portion 323b, and at the same time, the pressing portion 332 is pushed against the biasing force of the spring 331, and the spring 331 is loaded.

On the other hand, when the spindle unit SP contracts from the bent state of the knee joint mechanism 130 and the knee joint mechanism 130 moves from the bent state to the stretched side, the contact position between the pressing portion 332 and the cam portion 323 moves from the large diameter outer peripheral portion 323b to the small diameter outer peripheral portion 323a, and at the same time, the accumulated force of the spring 331 acts via the pressing portion 332 and the link member 320 in a direction for make the spindle unit SP contract. As a result, the stretching assist mechanism 330 can assist the stretching of the knee joint mechanism 130 with the force accumulated when the knee joint mechanism 130 is bent.

Note that in the transmission T according to the fourth embodiment, an example of a configuration in which the intermittence units 211 and 221 are provided between the first shaft 181 and the gear 183 and 185 and the intermittence units 212 and 222 are not provided between the second shaft 182 and the gears 184 and 186 is described, but as the transmission T according to the third embodiment, the intermittence units 212 and 222 may be provided between the second shaft 182 and the gear 184 and 186, and the intermittence units 211 and 221 may not be provided between the first shaft 181 and the gear 183 and 185.

Although various embodiments have been described above with reference to the drawings, it is needless to say that the present invention is not limited to these examples. It is apparent that those skilled in the art may conceive of various modifications and changes within the scope described in the claims, and it is understood that such modifications and changes naturally fall within the technical scope of the present invention. In addition, respective constituent elements in the above-described embodiment may be freely combined without departing from the gist of the invention.

For example, in the above embodiments, although prosthetic leg devices (electric prosthetic leg) applied to a knee joint as embodiments of the joint device of the present invention are described, the present invention is not limited thereto, and may be a prosthetic limb device (electric prosthetic limb) applied to an elbow joint, and the attachment subject may be an animal other than a human, or a robot. When applied to the elbow joint, the below-knee member 110 in the above embodiments becomes a distal end side of the attachment subject with respect to the above-knee member 120, that is, a forearm.

The expansion-contraction device 140, the transmission T, the first intermittence mechanism 210 and the second intermittence mechanism 220 provided in the transmission T, the first operating mechanism 230 and the second operating mechanism 240 that switch the first intermittence mechanism 210 and the second intermittence mechanism 220 or the like in the above embodiments are not limited to being applied to the joint device, and may also be applied to a drive device of a moving body such as a vehicle, or may be applied to a drive device of a working machine such as a snow blower or a lawn mower.

In the present description, at least the following matters are described. Corresponding constituent elements and the like in the embodiments described above are shown in parentheses, but the present invention is not limited thereto.

(1) A joint device (electric prosthetic leg 1), including:
a first member (below-knee member 110);
a second member (above-knee member 120);
coupling portion (knee joint mechanism 130) configured to couple the first member and the second member such that angles formed between the first member and the second member are variable; and
an expansion-contraction device (expansion-contraction device 140) capable of varying the angles formed between the first member and the second member by expanding and contracting, in which one side of the expansion-contraction device in an extending direction is mechanically connected to the first member, and an other side of the expansion-contraction device is mechanically connected to the second member,
in which assuming that one side of one circumference centering on a coupling axis (pivoting portion 135) of the coupling portion and having angles formed between the first member and the second member is defined as a first formed angle (first formed angle θ1), and an other side of the one circumference is defined as a second formed angle (second formed angle θ2), and
smaller one of minimum formed angles, of the first formed angle and the second formed angle, formed in a range of relative movement between the first member and the second member is defined as the second formed angle,
at least a part of the expansion-contraction device is disposed on a side of the first formed angle.

According to (1), the coupling portion can be stretched and bent by the expansion-contraction device. Since a side of the second formed angle is the side having the minimum formed angle, the setting range of the minimum formed angle on the side of the second formed angle can be made smaller by disposing at least a part of the expansion-contraction device on the side of the first formed angle, which is opposite to the second formed angle.

(2) The joint device according to (1),
in which the expansion-contraction device is provided such that a length of the expansion-contraction device in the extending direction decreases as the second formed angle increases.

According to (2), when the second formed angle increases, the length of the expansion-contraction device in the extending direction decreases (contracts). As a result, the joint device is bent.

(3) The joint device according to (1) or (2),
in which the expansion-contraction device includes:
   a power source (motor M) that outputs rotational power; and
   a motion conversion mechanism (spindle unit SP) that converts the rotational power output from the power source into translational motion.

According to (3), the power from the power source is output to the motion conversion mechanism, and the angles formed between the first member and the second member can be varied.

(4) The joint device ac-cording to (3),
in which the motion conversion mechanism is disposed on the side of the first formed angle.

According to (4), since the long motion conversion mechanism is disposed on the side of the first formed angle, the setting range of the minimum formed angle on the side of the second formed angle can be made smaller.

(5) The joint device according to (3) or (4),
in which the motion conversion mechanism includes:
   a shaft member (spindle 173); and
   a cylindrical member (sleeve 174) that performs translational motion along an axis of the shaft member due to a rotation of the shaft member.

According to (5), the motion conversion mechanism can be implemented with a simple configuration.

(6) The joint device according to (5),
in which the expansion-contraction device is provided such that a tensile force acts on the shaft member as the second formed angle increases.

According to (6), it is possible to prevent buckling deformation of the expansion-contraction device when the second formed angle increases.

(7) The joint device according to any one of (3) to (6),
in which the power source is disposed on a side of the second formed angle.

According to (7), the size of the power source can be adjusted according to the setting range of the minimum formed angle on the side of the second formed angle.

(8) The joint device according to any one of (3) to (7), further including:
a power storage device that supplies electric power to the power source,
in which the power storage device is disposed on the side of the first formed angle.

According to (8), the power storage device can be appropriately disposed.

(9) The joint device according to (8),
in which the motion conversion mechanism is disposed on the side of the first formed angle, and
the power source is disposed on the side of the second formed angle.

According to (9), the power storage device, the motion conversion mechanism, and the power source can be arranged in a well-balanced manner.

(10) A joint device (electric prosthetic leg 1), including:
a first member (below-knee member 110);
a second member (above-knee member 120);
a coupling portion (knee joint mechanism 130) configured to couple the first member and the second member such that angles formed between the first member and the second member are variable; and
an expansion-contraction device (expansion-contraction device 140) capable of varying the angles formed between the first member and the second member by expanding and contracting, in which one side of the expansion-contraction device in an extending direction is mechanically connected to the first member, and an other side of the expansion-contraction device is mechanically connected to the second member,
in which the expansion-contraction device has a motion conversion mechanism (spindle unit SP) including a shaft member (spindle 173) and a cylindrical member (sleeve 174) that performs translational motion along an axis of the shaft member due to a rotation of the shaft member, and
in which assuming that one side of one circumference centering on a coupling axis of the coupling portion and having angles formed between the first member and the second member is defined as a first formed angle (first angle θ1), and an other side of the one circumference is defined as a second formed angle (second formed angle θ2), and
smaller one of minimum formed angles, of the first formed angle and the second formed angle, formed in a range of relative movement between the first member and the second member is defined as the second formed angle,
the expansion-contraction device is provided such that a tensile force acts on the shaft member as the second formed angle increases.

According to (10), the coupling portion can be stretched and bent by the expansion-contraction device. It is also possible to prevent buckling deformation of the expansion-contraction device when the second formed angle increases.

(11) The joint device according to any one of (1) to (10),
in which the joint device is a prosthetic limb device configured to be attached to an attachment subject such that the first member is positioned on a distal end side of the attachment subject with respect to the second member.

According to (11), the joint device can be used as the prosthetic limb device.

(12) The joint device according to (11),
in which the prosthetic limb device is a prosthetic leg device configured to be attached to a leg portion of the attachment subject.

According to (12), the joint device can be used as a prosthetic leg device.

(13) The joint device according to (12),
in which the second member is configured to be attached to a thigh portion of the leg portion, and
the coupling portion is provided functioning as a knee joint between the thigh portion and a lower leg portion.

According to (13), the joint device can be used as a knee joint.

Although various embodiments have been described above with reference to the drawings, it is needless to say that the present invention is not limited to these examples. It is apparent that those skilled in the art may conceive of various modifications and changes within the scope described in the claims, and it is understood that such modifications and changes naturally fall within the technical scope of the present invention. In addition, respective constituent elements in the above-described embodiment may be freely combined without departing from the gist of the invention.

The present application is based on Japan patent application (Patent Application No. 2021-098286) filed on Jun. 11, 2021, and the contents of which are incorporated herein by reference.

### REFERENCE SIGNS LIST

1 electric prosthetic leg (joint device)
110 below-knee member (first member)
120 above-knee member (second member)
130 knee joint mechanism (coupling portion)
140 expansion-contraction device
173 spindle (shaft member)
174 sleeve (cylindrical member)
181 first shaft (third rotating body, seventh rotating body)
182 second shaft (fourth rotating body, eighth rotating body)
183 first drive gear (first rotating body)
184 first driven gear (second rotating body)
185 second drive gear (fifth rotating body)
186 second driven gear (sixth rotating body)
210 first intermittence mechanism
211 first intermittence unit
212 second intermittence unit
220 second intermittence mechanism
221 third intermittence unit
222 fourth intermittence unit
241 second operating rod (operation unit, operator)
241c large diameter portion (extending portion)
241c1 first large diameter portion (first extending portion, second extending portion)
241c2 second large diameter portion (first extending portion, second extending portion)
241c3 third large diameter portion (first extending portion, second extending portion)
242 second servomotor (drive unit)
271 roller (engager, engagement body)
272 pin (operation unit, actuator, advancing and retreating piece)
274 retainer (operation unit, actuator, engager)
281 roller (engager, engagement body)
282 retainer (operation unit, actuator, engager)
282c rubber ball (interposed member)
282d O-ring (interposed member)
283 pin (operation unit, actuator, advancing and retreating piece)
284 guide (operation unit, actuator)
S2 second internal space
T transmission
T1 first transmission mechanism
T2 second transmission mechanism
SP spindle unit (motion conversion mechanism)

1. A joint device, comprising:
a first member;
a second member;
a coupling portion configured to couple the first member and the second member such that angles formed between the first member and the second member are variable; and
an expansion-contraction device capable of varying the angles formed between the first member and the second member by expanding and contracting, in which one side of the expansion-contraction device in an extending direction is mechanically connected to the first member, and an other side of the expansion-contraction device is mechanically connected to the second member,
wherein assuming that one side of one circumference centering on a coupling axis of the coupling portion and having angles formed between the first member and the second member is defined as a first formed angle, and an other side of the one circumference is defined as a second formed angle, and
smaller one of minimum formed angles, of the first formed angle and the second formed angle, formed in a range of relative movement between the first member and the second member is defined as the second formed angle,
at least a part of the expansion-contraction device is disposed on a side of the first formed angle.

2. The joint device according to claim 1,
wherein the expansion-contraction device is provided such that a length of the expansion-contraction device in the extending direction decreases as the second formed angle increases.

3. The joint device according to claim 1,
wherein the expansion-contraction device includes:
a power source that outputs rotational power; and
a motion conversion mechanism that converts the rotational power output from the power source into translational motion.

4. The joint device according to claim 3,
wherein the motion conversion mechanism is disposed on the side of the first formed angle.

5. The joint device according to claim 3,
wherein the motion conversion mechanism includes:
a shaft member; and
a cylindrical member that performs translational motion along an axis of the shaft member due to a rotation of the shaft member.

6. The joint device according to claim 5,
wherein the expansion-contraction device is provided such that a tensile force acts on the shaft member as the second formed angle increases.

7. The joint device according to claim 3,
wherein the power source is disposed on a side of the second formed angle.

8. The joint device according to claim 3, further comprising:
a power storage device that supplies electric power to the power source,
wherein the power storage device is disposed on the side of the first formed angle.

9. The joint device according to claim 8,
wherein the motion conversion mechanism is disposed on the side of the first formed angle, and
the power source is disposed on a side of the second formed angle.

10. A joint device, comprising:
a first member;
a second member;
a coupling portion configured to couple the first member and the second member such that angles formed between the first member and the second member are variable; and
an expansion-contraction device capable of varying the angles formed between the first member and the second member by expanding and contracting, in which one side of the expansion-contraction device in an extending direction is mechanically connected to the first member, and an other side of the expansion-contraction device is mechanically connected to the second member,
wherein the expansion-contraction device has a motion conversion mechanism including a shaft member and a cylindrical member that performs translational motion along an axis of the shaft member due to a rotation of the shaft member, and
wherein assuming that one side of one circumference centering on a coupling axis of the coupling portion and having angles formed between the first member and the second member is defined as a first formed angle, and an other side of the one circumference is defined as a second formed angle, and
smaller one of minimum formed angles, of the first formed angle and the second formed angle, formed in a range of relative movement between the first member and the second member is defined as the second formed angle,
the expansion-contraction device is provided such that a tensile force acts on the shaft member as the second formed angle increases.

11. The joint device according to claim 1,
wherein the joint device is a prosthetic limb device configured to be attached to an attachment subject such that the first member is positioned on a distal end side of the attachment subject with respect to the second member.

12. The joint device according to claim 11,
wherein the prosthetic limb device is a prosthetic leg device configured to be attached to a leg portion of the attachment subject.

13. The joint device according to claim 12,
wherein the second member is configured to be attached to a thigh portion of the leg portion, and
the coupling portion is provided functioning as a knee joint between the thigh portion and a lower leg portion.

14. The joint device according to claim 10,
wherein the joint device is a prosthetic limb device configured to be attached to an attachment subject such that the first member is positioned on a distal end side of the attachment subject with respect to the second member.

15. The joint device according to claim 14,
wherein the prosthetic limb device is a prosthetic leg device configured to be attached to a leg portion of the attachment subject.

16. The joint device according to claim 15,
wherein the second member is configured to be attached to a thigh portion of the leg portion, and
the coupling portion is provided functioning as a knee joint between the thigh portion and a lower leg portion.
